(19) United States
(12) Patent Application Publication
(10) Pub. No.: US 2011/0015154 A1
(43) Pub. Date: Jan. 20, 2011

(54) SUPPORTING ACETYLCHOLINE FUNCTION

(76) Inventors: Gottfried H. Kellermann, Osceola, WI (US); Michael J. Bull, Somerset, WI (US)

Correspondence Address: FISH & RICHARDSON P.C. (TC), PO BOX 1022, MINNEAPOLIS, MN, 55440-1022, US

(21) Appl. No.: 12/839,475

(22) Filed: Jul. 20, 2010

Related U.S. Application Data
(60) Provisional application No. 61/226,979, filed on Jul. 20, 2009.

(51) Int. Cl. A61K 31/685; A61P 25/00; A61P 37/00

(52) U.S. Cl. 514/77

(57) ABSTRACT

This document provides methods and materials related to regulating inflammatory pathways. For example, compositions and kits containing two or more of an anticholinesterase compound, a choline compound, and a carnitine compound and methods for using the compositions and kits described herein to support acetylcholine function to regulate one or more inflammatory pathways are provided.

### CROSS-REFERENCE TO RELATED APPLICATIONS

This application claims priority under 35 U.S.C. 119(e) to U.S. Provisional Application Ser. No. 61/226,979, filed on Jul. 20, 2009, which is incorporated by reference in its entirety herein.

### BACKGROUND

1. Technical Field

This document provides methods and materials related to regulating inflammatory pathways. For example, compositions and kits containing two or more of an anticholinesterase compound, a choline compound, and a carnitine compound and methods for using the compositions and kits described herein to regulate one or more inflammatory pathways are provided.

2. Background Information

Inappropriate activation of inflammatory responses is the underlying cause of many common diseases. Recent studies have begun to elucidate the essential roles that acetylcholine and vagus nerve signaling play in controlling cytokine production as a way to limit or prevent damage from excess inflammation. Pavlov et al., Mol. Med., 9: 125-134. (2003); Tracey, J. Clin. Invest., 117: 289-296 (2007); Wang et al., Nature, 421: 384-388 (2003); Wang et al., Nat. Med., 10: 1216-1221 (2004). The cholinergic anti-inflammatory pathway is essential to the control of cytokine production to limit or prevent damage from excess inflammation. Tracey, J. Clin. Invest, 117: 289-296 (2007); Tracey, Nature, 420: 853-859 (2002).

Huperzine A, a compound found in the plant firmoss Huperzia serrata, is a reversible cholinesterase inhibitor known to increase acetylcholine levels in the brain and periphery. Liang & Tang, Acta Pharmacol. Sin., 27: 1127-1136 (2006). A recent study reported that after 14 days of huperzine A administration, nuclear translocation of transcription factor nuclear factor-kappa B (NFκB) was inhibited, overexpression of pro-inflammatory mediators in an immune challenge was decreased, and activation of glial cells in an ischemic event was reduced. Wang et al., J. Neurochem., 106: 1594-1603 (2008). Most actions of huperzine A are mediated by the cholinergic anti-inflammatory pathway, however, huperzine A also has non-cholinergic actions. Research has shown that huperzine A protects cells against hydrogen peroxide, beta-amyloid protein, glutamate, ischemia, staurosporine-induced cytotoxicity, and apoptosis, making it potentially useful to decrease oxidative stress and cognitive deficits such as dementia, mild cognitive impairment, and Alzheimer's disease Li et al., Cochrane. Database. Syst. Rev., CD005592 (2008); Wang et al., Acta Pharmacol. Sin., 27: 1-26 (2006). Cholinesterase inhibitors may decrease circulating norepinephrine levels. Peskind et al., Biol. Psychiatry, 38: 532-538 1995.

Alpha-glycerylphosphorylcholine (α-GPC) is known to be rapidly metabolized into choline and glycerophosphate and can cross the blood-brain barrier to enhance acetylcholine and phophatidylcholine biosynthesis, respectively. Abbiati et al., J. Chromatogr., 566: 445-451 (1991). α-GPC can be effective in the enhancement of CNS acetylcholine levels and may improve cognitive function in elderly patients with memory deficits (Canal et al., 1991; Di et al., 1991). With aging, the acetylcholine muscarinic M1 receptor density is decreased, and can lead to altered cell signaling and memory loss (Tayebati et al., 2002). In adult rats, α-GPC treatment countered the loss of M1 receptors but had no effect on M2 receptors. Amenta et al., Ann. N.Y. Acad. Sci., 695: 311-313 (1993). α-GPC may be beneficial not only for acetylcholine synthesis but also membrane receptor expression which can contribute to its cognitive enhancing effects. Drago et al., Pharmacol. Biohem. Behav., 41: 445-448 (1992).

Carnitine is an amino acid that promotes the transformation of free long-chain fatty acids into acylcarnitines with subsequent transport from the cytosol into the mitochondrial matrix, where they undergo β-oxidation for cellular energy production. Vaz & Wanders, Biochem. J., 361: 417-429 (2002). Carnitine is synthesized from the amino acids lysine and methionine and found in highest concentrations in heart and skeletal muscle. Bremer, Physiol Rev., 63:1420-1480 (1983). Studies have shown that carnitine can prevent the formation of reactive oxygen species, can scavenge free radicals, and can protect cells from peroxidative stress. Dokmeci, Folia Med. (Plovdiv.), 47:26-30 (2005); Zanelli et al., Ann. N.Y. Acad. Sci., 1053: 153-161 (2005). Although primary deficiency of carnitine is unusual, depletion can occur due to secondary causes, such as disease or a medication side effect. Carnitine deficiencies have been observed during dialysis in chronic renal failure, intestinal resection, severe infection, liver disease, cancer, diabetes, Alzheimer's disease, and heart failure. Evangeliou & Vlassopoulos, Curr. Pharm. Biotechnol., 4: 211-219 (2003); Cruciani et al., Ann. N.Y. Acad. Sci., 1033: 168-176 (2004). Supplementation with the acetylated form of L-carnitine, acetyl-L-carnitine, has been shown to reduce pain in patients with diabetic peripheral neuropathy. Evans et al., Ann. Pharmacother., 42: 1686-1691 (2008). acetyl-L-carnitine may also benefit patients with Alzheimer's disease by promoting α-secretase activity and the release of non-amyloidogenic fragment instead of a toxic segment. Epis et al., Eur. J. Pharmacol., 597: 51-56 (2008). In elderly subjects, acetyl-L-carnitine supplementation may reduce both physical and mental fatigue and improve cognitive status and physical functions. Malaguarnera et al., Arch. Gerontol. Geriatr., 46:181-190 (2008).

### SUMMARY

This document provides methods and materials related to supporting acetylcholine function for regulating inflammatory pathways. For example, compositions or kits containing a combination of one or more of an anticholinesterase compound, a choline compound, or a carnitine compound and methods for using a composition or kit as described herein to regulate one or more of an inflammatory pathway are provided. In some cases, a composition or kit as described herein can be administered to a human to relieve excess inflammation (e.g., by decreasing the level of pro-inflammatory cytokines, chemokines, neurotransmitters, growth factors, and amino acids and/or increasing the level of anti-inflammatory cytokines, chemokines, neurotransmitters, growth factors, and amino acids).

A composition or kit as described herein can be used to reduce one or more pro-inflammatory pathways and enhance one or more anti-inflammatory pathways. In some cases, promoting a healthy inflammatory response can enhance the health or well-being of a human diagnosed with, or identified as having, various conditions in which dysregulation of inflammatory pathways has been implicated as a cause or symptom. In one embodiment, a composition or kit as described herein can be used by a human who is overweight or obese, or at risk of developing symptoms associated with an unhealthy weight, to facilitate weight loss or weight maintenance, curb appetite, reduce food cravings, maintain efficient metabolism of the body, support the nervous system to promote a positive mood, enhance a feeling of satiety, reduce Body Mass Index (BMI), interfere with unhealthy adipose cell signaling, and limit the risk of impaired glucose tolerance progressing to diabetes. In another embodiment, a composition or kit as described herein can be used by a human who has a sleep disorder, such as central, complete or partial obstructive sleep apnea to support airway opening by soothing local inflammation and enhancing sleep duration. In some cases, a composition or kit as described herein can be used by a human with a mood disorder, such as depression, anxiety, and panic attacks to relieve nervous symptoms including heart palpitations, sweating, trembling, and to ease anxiety, settle nerves, and restore calm. A composition or kit as described herein can be used by a human who has autism to enhance focus, to promote healthy neurotransmitter levels, to support a sense of well-being, and to maintain emotional and mental balance. In some cases, a composition or kit as described herein can be used by a human with coronary artery disease to control or minimize atherosclerosis, to support healthy circulation, to maintain regular heartbeat, and to support coronary artery integrity. In another embodiment, a composition or kit as described herein can be used by a human with a food allergy, such as gluten sensitivity or celiac disease, to support the health and integrity of mucous membranes of the bowels, to promote healthy absorption of nutrients in the intestines, to support healthy digestion, and to relieve dermatitis associated with gluten sensitivity. In some cases, a composition or kit as described herein can be used by a human with Lyme disease to support the immune system and to relieve joint pain and stiffness. In another embodiment, a composition or kit as described herein can be used by a human with a thyroid imbalance (e.g., hyperthyroidism) to support healthy thyroid functioning, to maintain production of thyroid hormone within normal limits, and to support balanced activity of the endocrine system, for example.

Provided herein is a composition having a therapeutically effective amount of two or more of an anticholinesterase compound, a choline compound, and a carnitine compound. For example, an anticholinesterase compound and a choline compound; an anticholinesterase compound a carnitine compound; a choline compound, and a carnitine compound; an anticholinesterase compound, a choline compound, and a carnitine compound.

The anticholinesterase compound can include huperzine A, or a pharmaceutically acceptable salt thereof, in an amount ranging from about 0.003 mg to 3.0 mg. The choline compound can include alpha-GPC, or a pharmaceutically acceptable salt thereof, in an amount ranging from 10 mg to 10,000 mg. The carnitine compound can include acetyl-L-carnitine, or a pharmaceutically acceptable salt thereof, in an amount ranging from 20.0 mg to 20,000 mg.

The compositions described herein can be provided as a kit having a therapeutically effective amount of two or more of an anticholinesterase compound, a choline compound, and a carnitine compound in separate dosage forms.

Further provided herein is a method of regulating one of more inflammatory pathways in a human, the method comprising administering to the human an effective amount of a composition as described herein. In some embodiments, the regulating one or more inflammatory pathways in the human can include altering a level of one or more inflammatory mediators. For example, cytokines, chemokines, growth factors, neurotransmitters, and amino acids. In some embodiments, the one or more inflammatory mediators are selected from the group consisting of IL-1α, IL-1β, IL-6, TNF-α, leukocyte inhibitory factor (LIF), INF-γ, ciliary neuronotrophic factor (CNTF), GM-CSF, IL-11, IL-12, IL-17, IL-18, IL-8, IL-4, IL-10, IL-13, IL-16, IFN-α, G-CSF, TGF-β, soluble receptors for TNF and IL-6, norepinephrine, epinephrine, serotonin, and dopamine. In some cases, the level of one or more pro-inflammatory mediators can be decreased relative to the level of anti-inflammatory mediators.

In some embodiments, the human is obese. In some embodiments, the human is identified as having one or more of the group selected from: autism, Lyme disease, irritable bowel syndrome, a food allergy, coronary artery disease, a mood disorder and hyperthyroidism.

Also provided herein is a method of treating obesity in a human, the method comprising administering to the human an effective amount of a composition as described herein. The compositions described herein can also be used to support a healthy body weight in a human.

The compositions as described herein can be used in a variety of applications, including treating autism; treating panic attacks treating sleep apnea; supporting healthy sleep patterns; treating coronary artery disease; supporting a healthy cardiovascular system; treating Lyme disease; treating irritable bowel syndrome; treating gluten sensitivity; promoting healthy digestive functioning; treating hyperthyroidism; restoring balanced activity of the thyroid gland; altering the level of a neurotransmitter; altering the level of a cytokine; altering the level of a growth factor; and treating excess inflammation in a human.

Further provided herein is a method of treating one or more of ADHD, ADD, long-term depression (‘dysthymic’ depression) and PTSD, the method comprising administering to the human a composition as described herein. In some embodiments, the composition supports central nervous system cholinergic activities that target the locus ceruleus and the rostral ventral lateral medulla of the brain stem and/or increases the activity of the HPA axis via stimulation of the LC/brainstem nuclei.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 depicts graphs of monoamine and metabolite changes over seven hours. The mean values for each time point were graphed. n=8, *pp<0.05, **p<0.01. Panel A, epinephrine; panel B, norepinephrine; panel C, dopamine; panel D, 3,4-dihydroxyphenylacetic acid (DOPAC); panel E, serotonin; panel F, 5-hydroxyindoleacetic acid (5-HIAA).

FIG. 2 depicts graphs of measured amino acid changes over seven hours. The mean values for each time point were graphed. n=8, *p<0.05, panel A, glycine; panel B, taurine; panel C, γ-Aminobutyric acid (GABA); panel D, glutamine; panel E, glutamate, panel F, aspartic acid.

FIG. 3 depicts graphs of transmitter and cortisol changes over seven hours. The mean values for each time point were graphed. Panel A, phenylethylamine (PEA); panel B, histamine; panel C, cortisol. n=8 for panels A and B, n=9 for panel C, *p<0.05.

FIG. 4 depicts graphs of pro-inflammatory cytokine changes after four hours and one week. The mean values for each time point were graphed, indicated with a straight line. n=7, *p<0.05, **p<0.01. Panel A, IL-1beta; panel B, IL-2; panel C, IL-6; panel D, IL-7; panel E, IL-8; panel F, IL-12.

FIG. 5 depicts graphs of pro-inflammatory cytokine changes after four hours and one week. The mean values for each time point were graphed, indicated with a straight line. n=7, *p<0.05, **p<0.01. Panel A, IL-17; panel B, INF-γ; panel C, TNF-α.

FIG. 6 depicts graphs of chemokine and growth factor changes after four hours and one week. The mean values for each time point were graphed, indicated with a straight line. n=7, *p<0.05, ** p<0.01. Panel A, Monocyte chemotactic protein-1 (MCP-1); Panel B, macrophage inflammatory protein beta (MIP-β); Panel C, Granulocyte colony-stimulating factor (G-CSF); Panel D, Granulocyte-macrophage colony-stimulating factor (GM-CSF).

FIG. 7 depicts graphs of anti-inflammatory cytokine changes after four hours and one week. The mean values for each time point were graphed, indicated with a straight line. n=7, *p<0.05. Panel A, IL-4; panel B, IL-5; panel C, IL-10; panel D, IL-13.

FIG. 8 depicts a time series of R-R intervals derived from an ECG. The time occurrence of the R peak is identified for each heartbeat (grey lines). The R-R integral is determined by the duration between consecutive R peaks.

FIG. 9 depicts a bar graph of the average R-R intervals determined at 0, 1, 2, 4, 5, 6, and 7 hours. (n=9)

FIG. 10 depicts a bar graph of the average changes in standard deviation of the normal-to-normal intervals (SDNN) determined at 0, 1, 2, 4, 5, 6, and 7 hours. ms=milliseconds. (n=9)

FIG. 11 depicts a bar graph of the average changes in root mean square of successive inter beat intervals (RMSSD) determined at 0, 1, 2, 4, 5, 6, and 7 hours. ms=milliseconds. (n=9)

FIG. 12 depicts graphs of monoamine and metabolite changes over seven days. The mean values for each time point were graphed, indicated with a straight line. n=7, **p<0.01. Panel A, epinephrine; panel B, norepinephrine; panel C, dopamine; panel D, 3,4-DOPAC; panel E, serotonin; panel F, 5-HIAA.

FIG. 13 depicts graphs of measured amino acid changes over seven days. The mean values for each time point were graphed, indicated with a straight line. n=7, *p<0.05, panel A, glycine; panel B, taurine; panel C, GABA; panel D, glutamine; panel E, glutamate, panel F, aspartic acid.

FIG. 14 depicts graphs of transmitter and cortisol changes over seven days. The mean values for each time point were graphed, indicated with a straight line. n=7, *p<0.05. Panel A, PEA; panel B, histamine; panel C, cortisol.

### DETAILED DESCRIPTION

This document provides methods and materials related to regulating one or more inflammatory pathways in a mammal. For example, compositions or kits containing a combination of one or more of an anticholinesterase compound, a choline compound, and a carnitine compound, and methods for using compositions and kits described herein to regulate inflammatory pathways are provided.

### Definitions

The term “support acetylcholine function” means any activity that maintains or increases the level of acetylcholine available to act on a cell. For example, inhibiting acetylcholine catabolism and/or enhancing acetylcholine anabolism can support acetylcholine function.

The term “regulating inflammatory pathways” means reducing excess inflammatory responses by, e.g., promoting an anti-inflammatory response, decreasing the level of pro-inflammatory mediators, and/or increasing the level of anti-inflammatory mediators.

The term “decreased level” as used herein with respect to the level of an inflammatory mediator is any level that is below a median level for that modulator in a biological fluid (e.g., urine, saliva, serum, and/or blood) from a random population of healthy humans (e.g., a random population of 10, 20, 30, 40, 50, 100, or 500 humans). A decreased level of an inflammatory mediator can also be any level that is below a baseline level (e.g., before administration of a composition of the invention) of that mediator as measured in a human subject. In some cases, a decreased level can be an undetectable level of that modulator in biological sample.

The term “increased level” as used herein with respect to the level of a inflammatory mediator is any level that is above a median level for that mediator in a biological fluid (e.g., urine, saliva, serum, and/or blood) from a random population of healthy humans (e.g., a random population of 10, 20, 30, 40, 50, 100, or 500 humans). An increased level of an inflammatory mediator can also be any level that is above a baseline level (e.g., before administration of a composition of the invention) of that mediator as measured in a human subject.

The terms “ameliorate” and “treat” are used interchangeably and include both therapeutic treatment and/or prophylactic treatment (reducing the likelihood of development). Both terms mean decrease, suppress, attenuate, diminish, arrest, or stabilize the development or progression of a disease (e.g., a disease or disorder delineated herein), lessen the severity of the disease or improve the symptoms associated with the disease.

“Disease” means any condition or disorder that damages or interferes with the normal function of a cell, tissue, organ, or organism.

A salt of a compound can be formed between an acid and a basic group of the compound, such as an amino functional group, or a base and an acidic group of the compound, such as a carboxyl functional group. A compound used in a composition or kit herein can be a salt, e.g., a pharmaceutically acceptable salt.

The term “pharmaceutically acceptable,” as used herein, refers to a component that is, within the scope of sound medical judgment, suitable for use in contact with the tissues of humans and other mammals without undue toxicity, irritation, allergic response and the like, and are commensurate with a reasonable benefit/risk ratio. A “pharmaceutically acceptable salt” means any suitable salt that, upon administration to a recipient, is capable of providing, either directly or indirectly, a compound as described herein. A “pharmaceutically acceptable counterion” is an ionic portion of a salt that is not toxic when released from the salt upon administration to a recipient.

Acids commonly employed to form pharmaceutically acceptable salts include inorganic acids such as hydrogen bisulfide, hydrochloric acid, hydrobromic acid, hydroiodic acid, sulfuric acid and phosphoric acid, as well as organic acids such as para toluenesulfonic acid, salicylic acid, tartaric acid, bitartaric acid, ascorbic acid, maleic acid, besylic acid, fumaric acid, gluconic acid, glucuronic acid, formic acid, glutamic acid, methanesulfonic acid, ethanesulfonic acid, benzenesulfonic acid, lactic acid, oxalic acid, para bromophenylsulfonic acid, carbonic acid, succinic acid, citric acid, benzoic acid and acetic acid, as well as related inorganic and organic acids. Such pharmaceutically acceptable salts thus include sulfate, pyrosulfate, bisulfate, sulfite, bisulfite, phosphate, monohydrogenphosphate, dihydrogenphosphate, metaphosphate, pyrophosphate, chloride, bromide, iodide, acetate, propionate, decanoate, caprylate, acrylate, formate, isobutyrate, caprate, heptanoate, propiolate, oxalate, malonate, succinate, suberate, sebacate, fumarate, maleate, butyne 1,4 dioate, hexyne 1,6 dioate, benzoate, chlorobenzoate, methylbenzoate, dinitrobenzoate, hydroxybenzoate, methoxybenzoate, phthalate, terephathalate, sulfonate, xylene sulfonate, phenylacetate, phenylpropionate, phenylbutyrate, citrate, lactate, 0 hydroxybutyrate, glycolate, maleate, tartrate, methanesulfonate, propanesulfonate, naphthalene β sulfonate, naphthalene 2 sulfonate, mandelate and other salts.

### Compositions And Kits

Compositions and kits described herein include an effective amount of two or more of an anticholinesterase compound, a choline compound, and a carnitine compound. Anticholinesterase, choline, and carnitine compounds can be used to support acetylcholine function by maintaining or increasing endogenous acetylcholine accumulation, biosynthesis, and activity. In some cases, two or more compounds can be admixed to result in a composition of a single dosage form. For example, compositions described herein can comprise an admixture of an anticholinesterase compound and a choline compound, an admixture of an anticholinesterase compound and a carnitine compound, an admixture of a choline compound and a carnitine compound, and an admixture of an anticholinesterase compound, a choline compound, and a carnitine compound in a single dosage form.

In an alternative embodiment, two or more of the compounds described above can be presented in a separate dosage form as a kit. The term “kit” as used herein means that the separate dosage forms are packaged together or otherwise associated with one another or attached to one another such that it is readily apparent that the separate dosage forms are intended to be sold and administered together (within less than 24 hours of one another, consecutively or simultaneously). For example, kits as described herein can include a separate dosage form of an anticholinesterase compound associated with a separate dosage form of a choline compound, a separate dosage form of an anticholinesterase compound associated with a separate dosage form of a carnitine compound, a separate dosage form of a carnitine compound associated with a separate dosage form of a choline compound, or a kit comprising an anticholinesterase compound, a choline compound, and a carnitine compound each in a separate dosage form.

An appropriate anticholinesterase compound for use in compositions and kits described herein can be a cholinesterase inhibitor, i.e. an agent that functions to inhibit enzymatic breakdown of acetylcholine (e.g., physostigmine, neostigmine, pyridostigmine, ambenonium, demarcarium, rivastigmine, phenanthrene derivatives, galantamine, donepezil, tacrine, edrophonium, huperzine A, and diisopropyl phosphorofluoridate). In some embodiments, an anticholinesterase compound can be huperzine A, which can be in present in leaves or the purified extract from Huperzia serrata (e.g., available from Sigma Chemical Co., U.S.A.).

An appropriate choline compound for use in compositions and kits described herein can be an acetylcholine precursor (e.g., centrophenoxine, dimethyl-amino ethanol (DMAE), cytidine 5′ diphosphatidylcholine (CDP-choline)), choline, or choline derivatives (e.g., Alpha-glycerylphosphorylcholine (α-GPC) alpha-phosphatidylcholine or lecithin) and can be used to support acetylcholine function by maintaining or increasing acetylcholine biosynthesis and activity. In some embodiments, a composition or kit as described herein can include α-GPC (alpha size or non-alpha size, available from ChemiNutra, White Bear Lake, Minn., for example).

An appropriate carnitine compound for use in compositions and kits described herein can be carnitine, acetyl-L-carnitine, acetyl L-carnitine arginine, acetyl L-carnitine hydrochloride, acetyl L-carnitine arginine dihydrochloride or propionyl-L-carnitine. In some embodiments, a composition or kit as described herein can include acetyl-L-carnitine.

Compositions and kits described herein comprise an effective amount of two or more of an anticholinesterase compound, a choline compound and a carnitine compound or a pharmaceutically acceptable salt of said compounds; and in some embodiments, an acceptable carrier. A composition is formulated for pharmaceutical use (“a pharmaceutical composition”), wherein the carrier is a pharmaceutically acceptable carrier. The carrier(s) are “acceptable” in the sense of being compatible with the other ingredients of the formulation and, in the case of a pharmaceutically acceptable carrier, not deleterious to the recipient thereof in an amount used in the composition.

Pharmaceutically acceptable carriers, adjuvants and vehicles that may be used in the compositions of this invention include, but are not limited to, ion exchangers, alumina, aluminum stearate, lecithin, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

If required, the solubility and bioavailability of the compounds of the present invention in the compositions may be enhanced by methods well-known in the art. One method includes the use of lipid excipients in the formulation. See “Oral Lipid-Based Formulations: Enhancing the Bioavailability of Poorly Water-Soluble Drugs (Drugs and the Pharmaceutical Sciences),” David J. Hauss, ed. Informa Healthcare, 2007; and “Role of Lipid Excipients in Modifying Oral and Parenteral Drug Delivery: Basic Principles and Biological Examples,” Kishor M. Wasan, ed. Wiley-Interscience, (2006). Another known method of enhancing bioavailability is the use of an amorphous form of a compound of this invention optionally formulated with a poloxamer, such as LUTROL™ and PLURONIC™ (BASF Corporation), or block copolymers of ethylene oxide and propylene oxide. See U.S. Pat. No. 7,014,866; and U.S. patent publications 20060094744 and 20060079502.

The compositions and kits as described herein include those suitable for oral, rectal, nasal, topical (including buccal and sublingual), vaginal or parenteral (including subcutaneous, intramuscular, intravenous and intradermal) administration. In certain embodiments, the composition or kit as described herein is administered transdermally (e.g., using a transdermal patch or iontophoretic techniques). Other formulations may conveniently be presented in unit dosage form, e.g., tablets, sustained release capsules, and in liposomes, and may be prepared by any methods well known in the art of pharmacy. See, for example, Remington's Pharmaceutical Sciences, Mack Publishing Company, Philadelphia, Pa. (17th ed. 1985).

In certain embodiments, the compound is administered orally. Compositions of the present invention suitable for oral administration may be presented as discrete units such as capsules, sachets, or tablets each containing a predetermined amount of the active ingredient; a powder or granules; a solution or a suspension in an aqueous liquid or a non-aqueous liquid; an oil-in-water liquid emulsion; a water-in-oil liquid emulsion; packed in liposomes; or as a bolus, etc. Soft gelatin capsules can be useful for containing such suspensions, which may beneficially increase the rate of compound absorption. In some embodiments, capsules for oral use can include vegetable cellulose, microcrystalline, or carob, which is void of any animal derivatives. The compositions and kits described herein can be hypoallergenic.

When aqueous suspensions are administered orally, the active ingredient may be combined with emulsifying and suspending agents. In some embodiments, a composition or kit as described herein can be mixed with soft food (e.g., yogurt, pudding, apple sauce, oatmeal, or baby food) for oral administration. If desired, certain sweetening and/or flavoring and/or coloring agents may be added (e.g., fructose and lemon, rosemary, or peppermint oil). Compositions suitable for oral administration include lozenges comprising the ingredients in a flavored basis, such as sucrose and acacia or tragacanth; and pastilles comprising the active ingredient in an inert basis such as vegetable cellulose, gelatin and glycerin, or sucrose and acacia. Compositions suitable for parenteral administration include aqueous and non-aqueous sterile injection solutions which may contain anti-oxidants, buffers, bacteriostats and solutes which render the formulation isotonic with the blood of the intended recipient; and aqueous and non-aqueous sterile suspensions which may include suspending agents and thickening agents. The formulations may be presented in unit-dose or multi-dose containers, for example, sealed ampules and vials, and may be stored in a freeze dried (lyophilized) condition requiring only the addition of the sterile liquid carrier, for example water for injections, immediately prior to use. Extemporaneous injection solutions and suspensions may be prepared from sterile powders, granules and tablets.

Such injection solutions may be in the form, for example, of a sterile injectable aqueous or oleaginous suspension. This suspension may be formulated according to techniques known in the art using suitable dispersing or wetting agents (such as, for example, Tween 80) and suspending agents. The sterile injectable preparation may also be a sterile injectable solution or suspension in a non-toxic parenterally-acceptable diluent or solvent, for example, as a solution in 1,3-butanediol. Among the acceptable vehicles and solvents that may be employed are mannitol, water, Ringer's solution and isotonic sodium chloride solution. In addition, sterile, fixed oils are conventionally employed as a solvent or suspending medium. For this purpose, any bland fixed oil may be employed including synthetic mono- or diglycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant.

The compositions and kits described herein may be administered in the form of suppositories for rectal administration. These compositions can be prepared by mixing a compound of this invention with a suitable non-irritating excipient which is solid at room temperature but liquid at the rectal temperature and therefore will melt in the rectum to release the active components. Such materials include, but are not limited to, cocoa butter, beeswax and polyethylene glycols.

The compositions and kits described herein may be administered by nasal aerosol or inhalation. Such compositions are prepared according to techniques well-known in the art of pharmaceutical formulation and may be prepared as solutions in saline, employing benzyl alcohol or other suitable preservatives, absorption promoters to enhance bioavailability, fluorocarbons, and/or other solubilizing or dispersing agents known in the art. See, e.g., Rabinowitz and Zaffaroni, U.S. Pat. No. 6,803,031.

Topical administration of the compositions and kits described herein can be especially useful when the desired treatment involves areas or organs readily accessible by topical application. For topical application topically to the skin, the pharmaceutical composition should be formulated with a suitable ointment containing the active components suspended or dissolved in a carrier. Carriers for topical administration of the compositions and kits described herein can include, but are not limited to, mineral oil, liquid petroleum, white petroleum, propylene glycol, polyoxyethylene polyoxypropylene compound, emulsifying wax, and water. Alternatively, the pharmaceutical composition can be formulated with a suitable lotion or cream containing the active compound suspended or dissolved in a carrier. Suitable carriers include, but are not limited to, mineral oil, sorbitan monostearate, polysorbate 60, cetyl esters wax, cetearyl alcohol, 2-octyldodecanol, benzyl alcohol, and water. The pharmaceutical compositions of this invention may also be topically applied to the lower intestinal tract by rectal suppository formulation or in a suitable enema formulation. Topically-transdermal patches and iontophoretic administration are also included in this invention.

In another embodiment, a composition or kit as described herein further comprises a second therapeutic agent. The second therapeutic agent may be selected from any compound or therapeutic agent known to have or that demonstrates advantageous properties when administered with a compound having the same mechanism of action of an anticholinesterase compound, a choline compound, or a carnitine compound, e.g., a second anticholinesterase compound, choline compound, or carnitine compound as described above; an anti-inflammatory agent (e.g., non-steroidal anti-inflammatory drugs, broad spectrum chemokine inhibitors, fatty acids, and glucocorticoids); an anti-oxidant agent (e.g., catechins, resveratrol, flavonoids, carotenoids, glutathione, co-enzyme Q10, idebenone, and ubiquinone); a cholinomimetic agent (e.g., bethanechol, pilocarpine, and cevimeline), a botanical or botanical extract (Mucuna pruriens , Vicia faba, Griffonia simplicifolia, Boswellia serrata, Rhodiola rosea, green tea extracts such as EGCG, and Stevia rebaudiana); an amino acid or amino acid derivative (e.g., taurine, glycine, N-acetyl-cysteine, L-phenylalanine, D,L-phenylalanine, L-methionine, selenomethionine L-histidine, N-acetyl-tyrosine, L-glutamine, 5 hydroxytryptophan, L-theanine, and 4-amino-3-phenylbutyric acid); a nutritional or dietary supplement (e.g., inositol, creatinine, Krill oil, fish protein hydrolysase, lecithin or phosphatidylserine enriched soy lecithin, alpha-lipoic acid, docosahexaenoic acid, eicosapentaenoic acid, and alpha-linoleic acid); a mineral (e.g., calcium, magnesium, zinc, selenium, manganese, chromium, molybdenum, and iodine); a vitamin (e.g., vitamin A (beta carotene or retinal acetate), vitamin C (ascorbic acid), vitamin D (cholecalciferol), vitamin E (d-alpha tocopherol succinate), thiamine, riboflavin, niacin (niacinamide), vitamin B6 (pyroxidine HCl or pyrodoxal 5′ phosphate), vitamin B12, biotin, folic acid (folacin), and pantothentic acid (calcium pantothene)), growth factors, polypeptides, brain-derived neurotrophic factor, and ciliary neurotrophic factor.

In some cases, the second therapeutic agent is an agent useful in the treatment or prevention of a disease or condition selected from immune response disorders, including rheumatoid and other arthritic disorders, systemic lupus erythmatosus, systemic dematomyositis, psoriasis and other skin conditions, asthma, gluten sensitivity, and other allergic disorders, inflammatory bowel disease, autoimmune hematologic disorders, and acute exacerbations of multiple sclerosis, colitis, pancreatitis, ischemia reperfusion, Crohn's disease, atherosclerosis, diabetes, multiple sclerosis, and cerebral and myocardial ischemia, septic shock syndrome, sepsis, meningitis, and severe trauma; hyperthyroidism; emotional lability, panic disorder, and depression, neurological disorders and neurodegenerative diseases, such as, e.g., autism, Alzheimer's disease and multiple sclerosis; brain injuries, such as, e.g., stroke, traumatic brain injury, ischemic event, hypoxic event and neuronal death; disturbances of consciousness disorders; sleep disorders and obstructive sleep apnea; cardiovascular diseases, such as, e.g., coronary artery disease, peripheral vascular diseases, myocardial infarctions, and atherosclerosis; sympathetically mediated pain, such as, allodynia, hyperpathia, hyperalgesia, dysesthesia, paresthesia, deafferentation pain, and anesthesia dolorosa pain; Lyme disease; and metabolic disorders, e.g., insulin resistance and obesity. In some cases, the second therapeutic agent is an agent useful in the treatment or prevention of a disease or condition obesity, panic attacks, Lyme disease, coronary artery disease, sleep apnea, hyperthyroidism, gluten sensitivity, irritable bowel syndrome, dementia (e.g., vascular dementia), and age-related cognitive decline.

In another embodiment, the invention provides separate dosage forms of a compound of this invention and one or more of any of the above-described second therapeutic agents, wherein the compound and second therapeutic agent are associated with one another. The term “associated with one another” as used herein means that the separate dosage forms are packaged together or otherwise attached to one another such that it is readily apparent that the separate dosage forms are intended to be sold and administered together (within less than 24 hours of one another, consecutively or simultaneously).

In the compositions and kits of the invention, the compounds are present in an effective amount. As used herein, the term “effective amount” refers to an amount which, when administered in a proper dosing regimen, is sufficient to reduce or ameliorate the severity, duration or progression of the disorder being treated, prevent the advancement of the disorder being treated, cause the regression of the disorder being treated, enhance or improve the prophylactic or therapeutic effect(s) of another therapy, or to promote and maintain healthy immunoregulation.

The interrelationship of dosages for animals and humans (based on milligrams per meter squared of body surface) is described in Freireich et al., (1966) Cancer Chemother. Rep 50: 219. Body surface area may be approximately determined from height and weight of the subject. See, e.g., Scientific Tables, Geigy Pharmaceuticals, Ardsley, N.Y., 1970, 537.

An effective amount of a compound in a composition or kit described herein can range from 0.003 mg to 20,000 mg. For example, an effective amount of the anticholinesterase compound huperzine A can range from 0.003 mg to 3.0 mg, from 0.005 mg to 1.0 mg, from 0.01 mg to 0.8 mg, or from 0.02 mg to 0.4 mg. An effective amount of the choline compound α-GPC can range from 10 mg to 10,000 mg, 50 mg to 8,000 mg, 100 mg to 6,000 mg, or from 200 mg to 4000 mg. An effective amount of the carnitine compound acetyl-L-carnitine can range from 20.0 mg to 20,000 mg, 100 mg to 10,000 mg, 200 mg to 7,500 mg, or from 250 mg to 5,000 mg, inclusive. An effective amount can be given any number of times throughout the course of a day, for example, once, twice, or up to three times daily depending on various factors recognized by those skilled in the art.

Effective amounts will also vary, as recognized by those skilled in the art, depending on the diseases treated, the severity of the disease, the route of administration, the sex, age and general health condition of the subject, excipient usage, the possibility of co-usage with other therapeutic treatments such as use of other agents and the judgment of the treating physician, clinician, or other healthcare provider. For example, guidance for selecting an effective dose can be determined by reference to the pharmacokinetic information for huperzine A, α-GPC, and acetyl-L-carnitine. For compositions or kits that comprise a second therapeutic agent, an effective amount of the second therapeutic agent is between about 0.01% to 100% of the amount normally utilized in a monotherapy regime using just that agent. The normal monotherapeutic dosages of these second therapeutic agents are well known in the art. See, e.g., Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), each of which references are incorporated herein by reference in their entirety.

It is expected that some of the second therapeutic agents referenced above will act synergistically with the compounds of this invention. When this occurs, it will allow the effective amount of the second therapeutic agent and/or the compound of this invention to be reduced from that required in a monotherapy. This has the advantage of minimizing toxic side effects of either the second therapeutic agent of a compound of this invention, synergistic improvements in efficacy, improved ease of administration or use and/or reduced overall expense of compound preparation or formulation.

### Methods of Treatment

This disclosure provides methods of regulating inflammatory responses in a human by supporting acetylcholine function. The methods comprise administering to the human a composition or kit as described herein. The effect of a composition or kit as described herein on acetylcholine function can be monitored by assessing biomarkers of acetylcholine function, such as levels of immunoregulatory mediators (neurotransmitters, cytokines, chemokines, growth factors, and amino acids) or heart rate. The levels of immunoregulatory mediators can be assayed from a biological fluid (e.g., urine, blood, serum, cerebrospinal fluid (CSF), and saliva) as described in the Example provided. Any appropriate method can be used to obtain a biological sample from a mammal. For example, a blood sample can be obtained by peripheral venipuncture or finger stick, saliva and urine samples can be obtained using standard urine collection techniques, and CSF can be obtained via a lumbar puncture. A sample can be manipulated prior to being evaluated for the level of one or more inflammatory mediators.

Any appropriate method can be used to assess the level of a inflammatory mediator in a biological fluid (e.g., mass spectroscopy (MS), gas chromatography (GC), GC-MS, capillary electrophoresis (CE), CE/time-of-flight MS, CE with laser induced fluorescence detection (CE-LIF), high-performance liquid chromatography (HPLC), HPLC-amperometric detection, immunoassays (e.g., ELISA, and bead based protein assay), cytometry (e.g., Fluorescence assisted cell sorting), cytokine bioassays, and other clinical or biochemistry laboratory techniques. Levels of an inflammatory mediator can be determined in conjunction with a standard calibration curve that can be run in parallel with the samples of interest. Heart rate variability can be assessed using an ECG, for example. Any appropriate data processing software can be used to analyze the results.

In some cases, a composition or kit as described herein can be administered to a human to modulate an inflammatory response, e.g., decrease the level of pro-inflammatory cytokines, chemokines, neurotransmitters, and amino acids and/or increase the level of anti-inflammatory cytokines, chemokines, neurotransmitters, and amino acids. As used herein, the term “pro-inflammatory” describes an immunoregulatory mediator that promotes inflammation, e.g., IL-1α, IL-1β, IL-6, TNF-α, leukocyte inhibitory factor (LIF), INF-γ, ciliary neuronotrophic factor (CNTF), GM-CSF, IL-11, IL-12, IL-17, IL-18, and IL-8. As used herein, the term “anti-inflammatory” describes an immunoregulatory mediator that counteracts various aspects of inflammation, for example cell activation or the production of pro-inflammatory mediators. Examples of anti-inflammatory mediators include IL-4, IL-10, IL-13, IL-16, INF-α, G-CSF, TGF-β, and soluble receptors for TNF and IL-6. The type, duration, and the extent of cellular activities induced by a particular immunoregulatory mediator can be influenced by the nature of the target cell, the microenvironments of the cell, the presence of other immunoregulatory mediators, and the temporal sequence of several immunoregulatory mediators acting on the same cell. The net effect of an inflammatory response can be determined by the balance between pro-inflammatory mediators and anti-inflammatory mediators.

In another embodiment, a composition or kit as described herein can be administered to a human to alter the level of an immunoregulatory mediator, such as epinephrine, norepinephrine, dopamine, DOPAC, serotonin, 5-HIAA, DHEA, cortisol, melatonin, GABA, histamine, Beta-phenylethylamine (PEA), glutamate, glutamine, glycine, taurine, aspartic acid, tyramine, tryptamine, and pro-inflammatory and anti-inflammatory mediators described above.

In other embodiments, a composition or kit as described herein can be used to treat obesity. Treating obesity can include, independently, reducing body weight, preventing obesity in an overweight human, promoting loss of excessive fat (e.g., abdominal fat), reducing the Body Mass Index (BMI) (kg/m²), reducing symptoms associated with obesity (e.g., uncontrolled blood glucose levels, elevated blood pressure, and increased cholesterol levels), preventing progression of metabolic syndrome, and reducing levels of C-reactive protein in blood. In some cases, treating obesity can include reducing systemic inflammation (e.g., by reducing levels of IL-6, IL-8, TNF-α, MCP-1, IL-17, or IL-1β).

A composition or kit as described herein can be used to manage weight. Examples of managing weight can include, independently, supporting a healthy body weight, supporting healthy efforts to lose weight with balanced lifestyle, maintaining a healthy body image, maintaining efficient metabolism in the body, supporting the digestive system, promoting the natural breakdown of fats, supporting a balanced appetite, supporting healthy energy levels, promoting nutrient absorption, acting in a supporting capacity to balance mood, reducing or preventing food cravings and comfort eating, facilitating weight loss or weight maintenance, enhancing a feeling of satiety, reducing BMI, and interfering with unhealthy adipose cell signaling. In some cases, the compositions and kits described herein can manage weight by modulating an inflammatory response, e.g., by increasing or decreasing the level of IL-6, IL-8, TNF-α, MCP-1, IL-17, or IL-1β.

In another embodiment, a composition or kit as described herein can be used to treat mood disorders and panic attacks. For example, a composition or kit as described herein can be used to, independently, reduce mood swings, calm temper and agitation, balance extreme emotions, eliminate uncharacteristic behavior, reduce impulsivity, restore balanced moods, reduce irritability and moodiness, maintain normal serotonin levels, relieve symptoms of melancholy and weepiness, reduce feelings of sadness, support emotional wellness and health, support the nervous system, lessen common feelings of the blues, support a healthy motivated attitude, maintain a positive outlook, support a reasonable positive mental attitude, maintain a well-adjusted outlook and positive temperament, support healthy sleep patterns and a healthy balanced appetite, lift mood, promote an easy-going, positive emotional outlook, encourage increased energy levels, relieve fears associated with social situations or leaving familiar surroundings, alleviate the feeling of losing control, ease anxiety caused by wide open spaces or crowds, relieve nervous symptoms including heart palpitations, dry mouth, sweating, trembling, and shortness of breath, support the health of the nervous system, help maintain balanced emotions during everyday pressure, stress and common nervous tension, support healthy feelings of well-being, soothe the nerves, reduce the mental fear of stage fright and embarrassment, alleviate nerves associated with fear of public speaking, decrease negative thoughts, and promote a sense of peace. In some cases, the compositions and kits described herein can treat mood disorders or panic attacks by modulating an inflammatory response, e.g., by decreasing the level of IL-6, IL-1α, IL-1β, IL-8, MCP-1, MIP-1β, GM-CSF, IL-18, IL-2, TNF-α, or IFN-α.

In another embodiment, a composition or kit as described herein can be used to treat autism. For example, a composition or kit as described herein can be used to support an affected individual's ability to interact with the world around them, soothe nerves and control harmful behavior, support a naturally balanced mood, support emotional health and feelings of well-being, support the nervous system, support a reasonable positive mental attitude, maintain a well-adjusted outlook and positive temperament, support healthy neurotransmitters responsible for facilitating a calm mood, calm hyperactive children, improve concentration so children can focus, reduce impulsive and erratic behavior, alleviate behavioral problems, and reduce involuntary twitching, spasms or noises. In some cases, the compositions and kits described herein can treat an individual with autism by decreasing the level of TNF-α, IL-6, GM-CSF, INFγ, IL-8, or IL-12, or increasing the level of TGF-β1.

In another embodiment, a composition or kit as described herein can be used to treat obstructive sleep apnea (e.g., complete or partial sleep apnea) or hypopnea. Treating obstructive sleep apnea or hypopnea can include, independently, reducing the Apnea-Hypopnea Index (AHI) or Respiratory Disturbance Index (RDI) of a human, reducing the duration of cessation of breathing due to soft tissue obstruction (e.g., to less than 10 seconds), reducing swelling/inflammation in the tissues of the airway (e.g., muscles and/or other tissues), reducing symptoms associated with apnea or hypopnea (e.g., sleep fragmentation, high blood pressure, weight gain, and sleepiness or grogginess, during the day), improving breathing when in a supine position, reducing body weight, and quieting snoring. In some cases, the compositions and kits described herein can be used to treat obstructive sleep apnea or hypopnea by decreasing the level of IL-6, IL-8, and TNF-α.

A composition or kit as described herein can be used to support healthy sleep patterns, cycles, or behavior. For example, a composition or kit as described herein can be used to promote healthy respiration during sleep, promote respiratory functioning and health, maintain open airways and easy breathing, support respiratory calm and steady breathing, support the health of air passages, promote healthier sleeping patterns, enhance sleep duration, support balance in the respiratory system, and support health in the brain and nervous system. In some cases, the compositions and kits described herein can support healthy sleep patterns, cycles, or behavior by decreasing the level of IL-6, IL-8, and TNF-α.

In another embodiment, a composition or kit as described herein can be used to treat hyperthyroidism. Treating hyperthyroidism can include, independently, reducing the activity of the thyroid gland, alleviating symptoms associated with hyperthyroidism (e.g., weight loss, nervous or anxious feelings, tremors or shakiness, arrhythmia, tachycardia, difficulty sleeping or osteoporosis), reducing inflammation of the thyroid gland, reducing levels of circulating thyroid hormone, reducing pituitary stimulation, and preventing enlargement of the thyroid.

A composition or kit as described herein can be used to support balanced activity in the thyroid. For example, a composition or kit as described herein can be used to restore balanced activity of the thyroid, soothe the thyroid gland, support the production of the thyroid hormone within normal limits, help support healthy thyroid functioning, and support balance in the endocrine system. In some cases, the compositions and kits described herein can treat hyperthyroidism by decreasing the level of IL-8 and IL-6.

In another embodiment, a composition or kit as described herein can be used to treat Lyme disease. For example, a composition or kit as described herein can be used to maintain healthy, mobile joints and muscles, keep joints moving freely, support health in large joints and small joints of the hands, feet, toes, elbows and knees, maintain healthy cartilage and connective tissue, maintain a healthy immune system, support vitality and healthy energy levels, maintain healthy circulation and blood flow in the body, support cellular health, and reduce symptoms associated with Lyme disease. In some cases, the compositions and kits described herein can treat Lyme disease by modulating (e.g., decreasing or increasing) the level of TNF-β, IL-1β, IL-6, IL-8, IL-10, G-CSF, IFN-γ, or TNF-α, for example.

In another embodiment, a composition or kit as described herein can be used to treat food allergies, such as gluten sensitivity. For example, a composition or kit as described herein can be used to alleviate pain and discomfort during digestion, reduce gas buildup in the digestive tract, relieve discomfort associated with certain foods, relieve skin irritation, promote balance and calm in the digestive system, support health in the digestive system, promote healthy digestive and bowel functioning, support the integrity and health of the mucus membranes of the digestive system, promote healthy absorption of nutrients, and prevent damage to digestive system due to food allergies. In some cases, the compositions and kits described herein can treat food allergies by modulating (e.g., decreasing or increasing) the level of IL-8, IL-4, IFN-γ, IL-15, IL-17, IL-18, or IL-21.

In another embodiment, a composition or kit as described herein can be used to treat coronary artery disease. Treating coronary artery disease can include, independently, preventing accumulation of plaque in coronary arteries, reducing atherosclerosis, alleviating angina, preventing heart failure and arrhythmias, and lowering the risk of thrombosis. In some cases, treating coronary artery disease can include decreasing the level of IL-1, IL-6, IL-8, TNF-α or

MCP-1β, or increasing the level of TNF-β1.

A composition or kit as described herein can be used to support a healthy cardiovascular system. Supporting cardiovascular health can include maintaining blood pressure within the normal range, supporting balance in the cardiovascular system, supporting blood flow to the heart and extremities, supporting healthy pumping action of the heart, maintaining a regular heartbeat, promoting coronary artery health and integrity, supporting healthy energy levels and soothing nervous tension, supporting heart and blood vessel strength, preventing atherosclerosis, and reducing inflammation in coronary arteries. In some cases, a composition or kit as described herein can support or be used to support a healthy cardiovascular system by modulating (e.g., decreasing or increasing) the level of IL-1, IL-6, IL-8, TNF-α, MCP-1β, IL-2, IL-4, IL-12, IL-10, IL-18, C-reactive protein, and CD40 ligand, or increasing the level of TNF-β1.

In another embodiment, a composition or kit as described herein can be used to treat irritable bowel syndrome (IBS). Treating IBS can include managing IBS flare-ups, reducing inflammation in the digestive system, relaxing spasms in the muscles of the digestive tract, promoting healthy absorption of nutrients, and relieving abdominal cramps. In some cases, a composition or kit as described herein can be used to treat irritable bowel syndrome by increasing serotonin levels.

A composition or kit as described herein can support a healthy digestive system Supporting the health of the digestive system can include alleviating inflammation in the digestive system, promoting balance and calm in the digestive system, promoting healthy digestive and bowel functioning, and supporting the integrity and health of the mucus membranes of the digestive system. In some cases, supporting a healthy digestive system can include increasing serotonin levels.

In another embodiment, the disclosure provides a method of modulating the level of one or more neurotransmitters (e.g., serotonin and dopamine) that can be available to activate receptors in the brain or enterochromaffin cells, by administering a composition or kit as described herein. In some cases, the method relates to modulating the level of metabolites of one or more neurotransmitters (e.g., DOPAC and 5-HIAA).

In another embodiment, the disclosure provides a method of modulating the level of one or more inflammatory mediators (e.g., growth factors (Granulocyte-colony stimulating factor (G-CSF) and Granulocyte-macrophage colony stimulating factor (GM-CSF)), chemokines (e.g., chemokines monocyte chemoattractant protein-1 (MCP-1) and macrophage inflammatory protein-1 beta (MIP-1β)), amino acids (e.g., glycine, taurine, glutamate, and aspartate), and cytokines (e.g., interleukins IL-lbeta (IL-1β), IL-2, IL-4, IL-5, IL-6, IL-7, IL-8, IL-10, IL-12, IL-13, and IL-17) in a human by administering a composition or kit as described herein. In some cases, method includes decreasing the level of pro-inflammatory mediators or increasing the level of anti-inflammatory mediators.

According to another embodiment, the disclosure provides a method of treating a subject suffering from, or susceptible to, a disease or condition that is beneficially treated by of a compound of acetylcholine, precursor of acetylcholine, or an inhibitor of a cholinesterase comprising the step of administering to the subject a composition or kit as described herein.

Such diseases and conditions include, but are not limited to immune response disorders, including rheumatoid arthritis and other arthritic disorders, systemic lupus erythmatosus, systemic dematomyositis, psoriasis and other skin conditions, asthma, gluten sensitivities, and other allergic disorders, inflammatory bowel disease, autoimmune hematologic disorders, and acute exacerbations of multiple sclerosis, colitis, pancreatitis, ischemia reperfusion, Crohn's disease, atherosclerosis, diabetes, multiple sclerosis, and cerebral and myocardial ischemia, septic shock syndrome, sepsis, meningitis, and severe trauma; hyperthyroidism; emotional lability, panic disorder, and depression, neurological disorders and neurodegenerative diseases, such as, e.g., autism, Alzheimer's disease and multiple sclerosis; brain injuries, such as, e.g., stroke, traumatic brain injury, ischemic event, hypoxic event and neuronal death; disturbances of consciousness disorders; sleep disorders and obstructive sleep apnea; cardiovascular diseases, such as, e.g., coronary artery disease, peripheral vascular diseases, myocardial infarctions, and atherosclerosis; sympathetically mediated pain, such as, allodynia, hyperpathia, hyperalgesia, dysesthesia, paresthesia, deafferentation pain, and anesthesia dolorosa pain; Lyme disease; and metabolic disorders, e.g., insulin resistance and obesity. In some cases, the second therapeutic agent is an agent useful in the treatment or prevention of a disease or condition obesity, panic attacks, Lyme disease, coronary artery disease, sleep apnea, hyperthyroidism, gluten sensitivity, irritable bowel syndrome, ADHD, ADD, PTSD, or long-term depression.

Methods delineated herein also include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In another embodiment, any of the above methods of treatment comprises the further step of co-administering to the subject one or more additional second therapeutic agents. The choice of second therapeutic agent may be made from any second therapeutic agent known to be useful for co-administration with of an anticholinesterase compound, a choline compound, or a carnitine compound. The choice of second therapeutic agent is also dependent upon the particular disease or condition to be treated. Examples of second therapeutic agents that may be employed in the methods described above for use in combination compositions comprising a compound of this invention and a second therapeutic agent.

In particular, the combination therapies of this invention include co-administering to a subject in need thereof a composition or kit as described herein and an additional therapeutic agent as described above.

The term “co-administered” as used herein means that the additional second therapeutic agent may be administered together with a compound or compositions of this invention as part of a single dosage form (such as a composition of this invention comprising a compound of the invention and an second therapeutic agent as described above) or as separate, multiple dosage forms. Alternatively, the additional agent may be administered prior to, consecutively with, or following the administration of a compound or composition of this invention. In such combination therapy treatment, both the composition or kit as described herein and the second therapeutic agent(s) are administered by conventional methods. The administration of a composition or kit of this invention, comprising both a composition or kit as described herein and a second therapeutic agent, to a subject does not preclude the separate administration of that same therapeutic agent, any other second therapeutic agent or any compound of this invention to said subject at another time during a course of treatment.

Effective amounts of these second therapeutic agents are well known to those skilled in the art and guidance for dosing may be found in patents and published patent applications referenced herein, as well as in Wells et al., eds., Pharmacotherapy Handbook, 2nd Edition, Appleton and Lange, Stamford, Conn. (2000); PDR Pharmacopoeia, Tarascon Pocket Pharmacopoeia 2000, Deluxe Edition, Tarascon Publishing, Loma Linda, Calif. (2000), and other medical texts. However, it is well within the skilled artisan's purview to determine the second therapeutic agent's optimal effective-amount range.

In one embodiment, where a second therapeutic agent is administered to a subject, the effective amount of the composition or kit of this invention is less than its effective amount would be where the second therapeutic agent is not administered. In another embodiment, the effective amount of the second therapeutic agent is less than its effective amount would be where the composition or kit of this invention is not administered. In this way, undesired side effects associated with high doses of either agent may be minimized. Other potential advantages (including without limitation improved dosing regimens and/or reduced drug cost) will be apparent to those of skill in the art.

In yet another aspect, the invention provides the use of a composition or kit as described herein together with one or more of the above-described second therapeutic agents in the manufacture of a medicament, either as a single composition or as separate dosage forms, for treatment or prevention in a subject of a disease, disorder or symptom set forth above. Another aspect of the invention is a composition or kit as described herein for use in the treatment or prevention in a subject of a disease, disorder or symptom thereof delineated herein.

### Articles of Manufacture

The present disclosure also provides articles of manufacture for use with the compositions and kits described herein. These articles of manufacture comprise (a) a composition or kit comprising two or more of anticholinesterase compounds, a choline compound, and a carnitine compound provided as an admixture or as separate components in association as described herein, wherein the composition or kit is in a container; and (b) instructions describing a method of using the composition or kit to support acetylcholine function, e.g., as described above.

In another embodiment, articles of manufacture for use to regulate inflammatory pathways are provided. Regulating inflammatory pathways to ameliorate excess inflammation can be useful for treating a human suffering from, or susceptible to, a disease or condition described above. In some cases, an article of manufacture as described herein can be useful in treating obesity, hyperthyroidism, sleep disturbances, Lyme disease, autism, panic attacks and cardiovascular disease.

In another embodiment, an article of manufacture as described herein can be used to support, enhance, or maintain overall health in a human. For example, articles of manufacture to manage body weight, to support healthy sleep patterns, to support balanced activity in the thyroid gland, to support a healthy cardiovascular system, and to support a healthy digestive system are provided herein.

According to another embodiment, an article of manufacture can include instructions describing a method of using a composition or kit as described herein to treat obesity. In some cases, an article of manufacture can include information regarding the potential benefits associated with administration of a composition or kit as described herein to treat obesity, such as reducing body weight, preventing obesity in an overweight human, promoting loss of excessive fat (e.g., abdominal fat), reducing the BMI, reducing symptoms associated with obesity (e.g., uncontrolled blood glucose levels, elevated blood pressure, and increased cholesterol levels), preventing progression of metabolic syndrome, and reducing levels of C-reactive protein in blood.

An article of manufacture described herein can be used to manage body weight. For example, an article of manufacture can include instructions describing a method of using a composition or kit as described herein to manage weight. In some cases, an article of manufacture can include information regarding the potential benefits associated with administration of a composition or kit as described above (e.g., supporting a healthy body weight, supporting healthy efforts to lose weight along with a balanced lifestyle, maintaining healthy weight goals, maintaining efficient metabolism in the body, promoting the natural breakdown of fats, supporting a healthy balanced appetite, supporting healthy energy levels and nutrient absorption, reducing or preventing food cravings and comfort eating, supporting routine weight management and a healthy metabolism, facilitating weight loss or weight maintenance, maintaining efficient metabolism, promoting a positive mood, positive body image, and increased energy, enhancing the feeling of satiety, and reducing BMI,.

In another embodiment, articles of manufacture for use to treat mood disorders and panic attacks are provided. Instructions describing a method of using the composition to treat panic attacks can provide information regarding the benefits associated with a composition or kit as described herein when used to treat a mood disorder or panic attack. For example, a composition or kit as described herein may reduce mood swings, calm temper and agitation, balance extreme emotions, eliminate uncharacteristic behavior, reduce impulses, restore healthy, balanced moods, reduce irritability and moodiness, maintain normal serotonin levels, relieve symptoms of melancholy and weepiness, reduce feelings of sadness, support emotional wellness and health, support the nervous system, lessen feelings of “the blues,” support a healthy motivated attitude, support a reasonable positive mental attitude, maintain a positive or well-adjusted outlook and positive temperament, support healthy sleep patterns and a healthy balanced appetite, lift mood, promote an easy-going, positive emotional outlook, encourage increased energy levels, relieve fears associated with social situations or leaving familiar surroundings, alleviate the feeling of losing control, ease anxiety caused by wide open spaces or crowds, relieve heart palpitations, dry mouth, sweating, trembling, and shortness of breath, support the health of the nervous system, help maintain balanced emotions during everyday stress, support healthy feelings of well-being, soothe the nerves, reduce the fear of stage fright and embarrassment, alleviate nerves associated with fear of public speaking, decrease negative thoughts, and promote a sense of peace.

In yet another embodiment, articles of manufacture for use to treat a human who has been identified as having autism are provided. Instructions describing a method of using the composition to treat a human with autism can provide information regarding the benefits associated with a composition or kit as described herein when used to treat a human with autism.

For example, a composition or kit as described herein may support an affected individual's ability to interact with the world around them, soothe nerves and control harmful behavior, support a naturally balanced mood, support emotional health, support the nervous system, support a reasonable positive mental attitude, maintain a well-adjusted outlook and positive temperament, support healthy neurotransmitters responsible for facilitating a calm mood, calm hyperactive children, improve concentration so children can focus, reduce impulsive and erratic behavior, alleviate behavioral problems, and reduce involuntary twitching, spasms or noises.

According to another embodiment, an article of manufacture can include instructions describing a method of using a composition or kit as described herein to treat obstructive sleep apnea or hypopnea. In some cases, an article of manufacture can include information regarding the potential benefits associated with administration of a composition or kit as described herein to treat obstructive sleep apnea or hypopnea, such as reducing the AHI or RDI, reducing the duration of cessation of breathing due to soft tissue obstruction, reducing swelling/inflammation in the tissues of the airway, reducing symptoms associated with apnea or hypopnea (e.g., sleep fragmentation, high blood pressure, weight gain, and sleepiness or grogginess, during the day), improving breathing when in a supine position, reducing body weight, and quieting snoring.

In another embodiment, an article of manufacture for supporting healthy sleep patterns, cycles, or behavior is provided herein. For example, an article of manufacture as described herein can include instructions describing a method of using a composition or kit as described herein and can include information regarding the benefits associated with using the composition to support healthy sleep patterns, cycles, or behavior, such as promoting healthy respiration during sleep, promoting respiratory functioning and health, maintaining open airways and easy breathing, supporting respiratory calm and steady breathing, supporting the health of air passages, and supporting balance in the respiratory system, for example.

According to another embodiment, an article of manufacture can include instructions describing a method of using a composition or kit as described herein to treat hyperthyroidism. In some cases, an article of manufacture can include information regarding the potential benefits associated with administration of a composition or kit as described herein to treat hyperthyroidism, such as reducing the activity of the thyroid gland, alleviating symptoms associated with hyperthyroidism (e.g., weight loss, nervous or anxious feelings, tremors or shakiness, arrhythmia, tachycardia, difficulty sleeping or osteoporosis), reducing inflammation of the thyroid gland, reducing levels of circulating thyroid hormone, reducing pituitary stimulation, and preventing enlargement of the thyroid, for example.

In another embodiment, articles of manufacture for use to support balanced activity of the thyroid are provided. An article of manufacture can include information regarding the potential benefits associated with administration of a composition or kit as described herein to support balanced activity of the thyroid, such as soothing the thyroid gland, supporting the healthy production of thyroid hormone, helping support healthy thyroid functioning, and supporting systemic balance in the endocrine system responsible for maintaining body metabolism.

An additional embodiment provides articles of manufacture for use in treating a human who has Lyme disease. Instructions describing a method of using a composition or kit as described herein to treat symptoms of Lyme disease can provide information regarding the benefits associated with using the composition or kit to treat Lyme disease. For example, a composition or kit as described herein may maintain healthy, mobile joints and muscles, keep joints moving freely, support health in large joints and small joints of the hands, feet, toes, elbows and knees, maintain healthy cartilage and connective tissue, maintain a healthy immune system, support vitality and healthy energy levels, maintain healthy circulation and blood flow in the body, support cellular health and reduce symptoms associated with Lyme disease.

In another embodiment, articles of manufacture for use to treat food allergies, such as gluten sensitivity, are provided. Instructions describing a method of using a composition or kit as described herein to treat gluten sensitivity can provide information regarding the benefits associated with using the composition or kit to treat gluten sensitivity. For example, a composition or kit as described herein may alleviate pain and discomfort during digestion, reduce gas buildup in the digestive tract, relieve discomfort associated with certain foods, promote balance and calm in the digestive system, support health in the digestive system, promote healthy digestive and bowel functioning, support the integrity and health of the mucus membranes of the digestive system, promote healthy absorption of nutrients, and prevent damage to digestive system due to food allergies.

According to another embodiment, an article of manufacture can include instructions describing a method of using a composition or kit as described herein to treat coronary artery disease. In some cases, an article of manufacture can include information regarding the potential benefits associated with administration of a composition or kit as described herein to treat coronary artery disease, such as preventing the accumulation of plaque in coronary arteries, reducing atherosclerosis, alleviating angina, preventing heart failure and arrhythmias, and lowering the risk of thrombosis.

According to another embodiment, an article of manufacture can include instructions describing a method of using a composition or kit as described herein to support cardiovascular health. In some cases, an article of manufacture can include information regarding the potential benefits associated with administration of a composition or kit as described herein to support a healthy cardiovascular system, such as maintaining blood pressure within the normal range, supporting balance in the cardiovascular system, supporting blood flow to the heart and the extremities, supporting healthy pumping action of the heart, maintaining a regular heartbeat, promoting coronary artery health and integrity, supporting healthy energy levels and soothing common nervous tension, supporting blood vessel strength, preventing atherosclerosis, and reducing inflammation in coronary arteries, for example.

In another embodiment, an article of manufacture can include instructions describing a method of using a composition or kit as described herein to treat IBS. In some cases, an article of manufacture can include information regarding the potential benefits associated with administration of a composition or kit as described herein to treat IBS, such as managing irritable bowel syndrome flare-ups, reducing inflammation in the digestive system, relaxing spasms in the muscles of the digestive tract, promoting healthy absorption of nutrients, and relieving abdominal cramps.

According to another embodiment, an article of manufacture can include instructions describing a method of using a composition or kit as described herein to support a healthy digestive system. In some cases, an article of manufacture can include information regarding the potential benefits associated with administration of a composition or kit as described herein to support a healthy digestive system, such as alleviating inflammation in the digestive system, promoting balance and calm in the digestive system, promoting healthy digestive and bowel functioning, and supporting the integrity and health of the mucus membranes of the digestive system, for example.

The container may be any vessel or other sealed or sealable apparatus that can hold said pharmaceutical composition. Examples include bottles, ampules, divided or multi-chambered holders bottles, wherein each division or chamber comprises a single dose of said composition, a divided foil packet wherein each division comprises a single dose of said composition, or a dispenser that dispenses single doses of said composition. The container can be in any conventional shape or form as known in the art which is made of a pharmaceutically acceptable material, for example a paper or cardboard box, a glass or plastic bottle or jar, a re-sealable bag (for example, to hold a “refill” of tablets for placement into a different container), or a blister pack with individual doses for pressing out of the pack according to a therapeutic schedule. The container employed can depend on the exact dosage form involved, for example, a conventional cardboard box would not generally be used to hold a liquid suspension. It is feasible that more than one container can be used together in a single package to market a single dosage form. For example, tablets may be contained in a bottle, which is in turn contained within a box. In on embodiment, the container is a blister pack.

The articles of manufacture described herein may also comprise a device to administer or to measure out a unit dose of the pharmaceutical composition. Such device may include an inhaler if said composition is an inhalable composition; a syringe and needle if said composition is an injectable composition; a syringe, spoon, pump, or a vessel with or without volume markings if said composition is an oral liquid composition; or any other measuring or delivery device appropriate to the dosage formulation of the composition present in the kit.

In certain embodiment, the articles of manufacture described herein may comprise in a separate vessel of container a pharmaceutical composition comprising a second therapeutic agent, such as one of those listed above for use for co-administration with a compound of this invention.

### EXAMPLES

### Example 1

### Oral Administration of A Test Composition

### Methods

Eleven healthy human adults (6 female, 5 male), aged 24-45 years were administered doses of a test composition containing 800 mg AlphaSize™ 50P (50% α-GPC, ChemiNutra, White Bear Lake, Minn.), 200 μg huperzine A (Sigma Chemical Co., U.S.A.), and 1000 mg acetyl-L-carnitine (Sigma Chemical Co., U.S.A.), orally. Subjects adhered to a specific schedule on a “control day” (Day 0), on a “treatment day” (Day 1) and after 1 week (Day 8) with regards to food and the test composition ingestion, specimen collection, and heart rate variability (HRV) assessment. The effect of the test composition on acetylcholine activity was assessed by monitoring biomarkers including urinary neurotransmitters, salivary cortisol, serum cytokines, and heart rate. Subjects collected specimens and monitored HRV at 0 hour (baseline), 1 hour, 2 hours, 4 hours, 5 hours, 6 hours, and 7 hours on Day 0 and Day 1. On Day 0, subjects collected urine and saliva specimens and monitored HRV. The control data (Day 0) were used to account for changes in salivary and urinary parameters and HRV affected by circadian rhythm. On Day 1, the subjects collected urine, saliva, and serum specimens, monitored HRV, and took a test composition twice (after 0 hour and 4 hour specimen collection time points). The subjects took a test composition twice daily for six more days and then collected one urine, one saliva, and one serum specimen on the next day (Day 8). On Day 8, subjects collected specimens at the 0 hour time point. Regimented breakfast meals were eaten at one hour before collecting the first specimens on Day 0, Day 1, and Day 8 and consisted of a Quaker® Chewy Granola Bar (chocolate chip), 3.9 ounces of Musselman's® Natural Unsweetened Applesauce and a 6 ounce box of Juicy Juice® (orange tangerine) to control dietary protein intake that may effect urinary neurotransmitter levels. Other fluid intake was limited during the specimen collections on Day 0 and Day 1 to prevent dilution of the urine specimens. Lunch meals were eaten on Day 0 and Day 1 at the 3 hour time point, which consisted of a package of Uncle Ben's® Ready Rice (Whole Grain Brown). Subjects noted subjective perceived changes in physiological or psychological function throughout the study. Two subjects deviated from the protocol, and these data were excluded analysis. An additional two subjects did not complete the 1 week dosing schedule for the test composition and no data was collected for these subjects on Day 8.

All urine, saliva, and serum specimens were analyzed by Pharmasan Labs, Inc. (Osceola, Wis.). Urine specimens were assessed for epinephrine, norepinephrine, dopamine, 3,4-dihydroxyphenylacetic acid (DOPAC), serotonin, 5-hydroxyindoleacetic acid (5-HIAA), glycine, taurine, gamma-aminobutyric acid (GABA), glutamine, glutamate, aspartic acid, phenylethylamine (PEA), and histamine. Additionally, creatinine levels for each sample were measured and neurotransmitters values were reported in parts per gram creatinine (/gCr). The hormone cortisol was assessed in the saliva specimens. Several cytokines, including the interleukins IL-1beta (IL-1(3), IL-2, IL-4, IL-5, IL-6, IL-7, IL-8, IL-10, IL-12, IL-13, and IL-17; the chemokines monocyte chemoattractant protein-1 (MCP-1) and macrophage inflammatory protein-1 beta (MIP-1β); and other cytokines such as granulocyte-colony stimulating factor (G-CSF, granulocyte-macrophage colony-stimulating factor (GM-CSF), IFN-γ, and tumor necrosis factor-alpha (TNF-α) were assessed in the serum specimens. Data were stored and organized using Excel® software (Microsoft® Corporation, Redmond, Wash.). Graphing and statistical analyses of the data were performed using Prism® 5 software (GraphPad Software, Inc., LaJolla, Calif.).

HRV was assessed with a single-lead ECG monitor (CheckMyHeart, Daily Care Biomedical, Chungli, Taiwan). Nine subjects (5 female, 4 male) took turns sitting in a chair in a quiet room with a researcher present and concentrated on a black circle drawn in the center of a white piece of paper. One ECG conductive adhesive electrode each was placed on the subject's right and left anterior forearms. Measurements were taken for 300 seconds (five minutes) at 250 Hz sampling rate on Day 0 and on Day 1, according to the schedule described above. The ECG data was analyzed using CheckMyHeart software (Daily Care Biomedical, Chungli, Taiwan) to calculate HRV. HRV was analyzed and graphed using Prism 5 software (GraphPad Software, Inc., LaJolla, Calif.). Two-way ANOVAs were performed to determine whether the treatment caused statistically significant changes in heart rate variability compared to the control day.

### Results

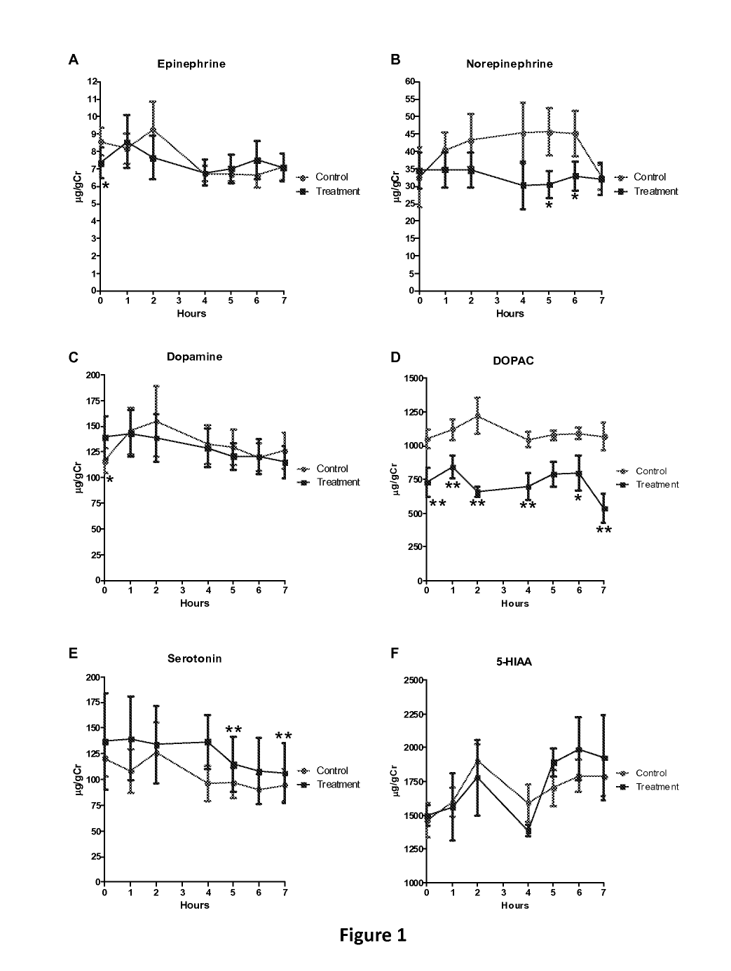

To investigate whether treatment with a specific test composition aimed at increasing acetylcholine affected neurotransmitter and cortisol levels, subjects collected baseline urine and saliva specimens (0 hour), ingested a test composition, collected urine and saliva specimens at 1 hour, 2 hours, and 4 hours after ingestion, took the test composition again, and collected urine and saliva specimens at 5 hours, 6 hours, and 7 hours (after initial ingestion). The mean values with the standard error of the mean (SEM) for each neurotransmitter and cortisol were graphed for each time point (FIGS. 1, 2, 3) for both the control day and treatment day. Although nine subjects completed the study, the sample size was n=8 for all urinary and salivary parameters due to removal of one subject's data because the specimens were too dilute to analyze properly. For the time points 5 hours, 6 hours, and 7 hours, the sample size was n=7 because one subject did not ingest the second dose on Day 1. Statistical analysis was performed using the one-tailed t-test on the means of paired samples comparing control data to treatment data.

### Neurotransmitters And Cortisol

Since the activity or levels of acetylcholine cannot be assessed directly, peripheral biomarkers were analyzed to indirectly monitor the sympathetic and parasympathetic effects of the test composition.

Support of acetylcholine pathways may result in increased epinephrine release and decreased norepinephrine. Pavlov et al., Mol. Med., 9: 125-134 (2003); Lechin & van der Dijs, Dig. Dis. Sci., 54:458-470 (2009). Epinephrine, showed a slight increase in mean values at 1, 5, and 6 hours after treatment when compared to control data, but there was a slight decrease at 0 hour and 2 hours (FIG. 1, A). Statistical significance was only observed at the 0 hour time point (p<0.05; Table 1). The mean values for norepinephrine levels were decreased at 1, 2, 4, 5, and 6 hours after treatment with statistical significance observed at 5 and 6 hours (FIG. 1, B; Table 1).

With enhanced parasympathetic activity, more serotonin is released from the enterochromaffin cells (Lechin & van der Dijs, Dig. Dis. Sci., 54:458-470 (2009)), so increased levels may be observed in the urine. Serotonin levels were increased at all time points with treatment (FIG. 1, E) and statistical significance was observed at 5 and 7 hours (p<0.01; Table 1).

Midbrain acetylcholine from the ventral tegmental area stimulates the release of dopamine from the nucleus accumbens and may possibly increase dopamine levels in the urine. Lester et al., Neuroreport, 19: 991-995 (2008). Dopamine levels showed slightly lower values for the treatment condition for all the time points, but statistical significance was only observed at 0 hour (FIG. 1, C; p<0.05, Table 1).

To determine whether the test composition alters the availability of monoamines inside a neuron or the exposure of monoamines to monoamine oxidase (MAO), thereby increasing the levels of metabolites of dopamine and serotonin, DOPAC and 5-HIAA were measured. DOPAC levels were decreased at all time points on the treatment day (FIG. 1, D). Statistically significant differences were seen at 0, 1, 2, 4, 6 and 7 hours (p<0.05 and p<0.01; Table 1). 5-HIAA levels decreased at 1, 2, and 4 hours, but increased at 5, 6 and 7 hours (FIG. 1, F); however, significant differences between treatment and control were not observed (Table 1). The increase in serotonin did not correspond with an increase in 5-HIAA, suggesting that synthesis of serotonin or the neuronal levels of serotonin were not increased. The decrease in urinary DOPAC levels did not correspond with decreased urinary dopamine. MAO is probably not affected by the test composition as the decrease in DOPAC did not result in increased dopamine.

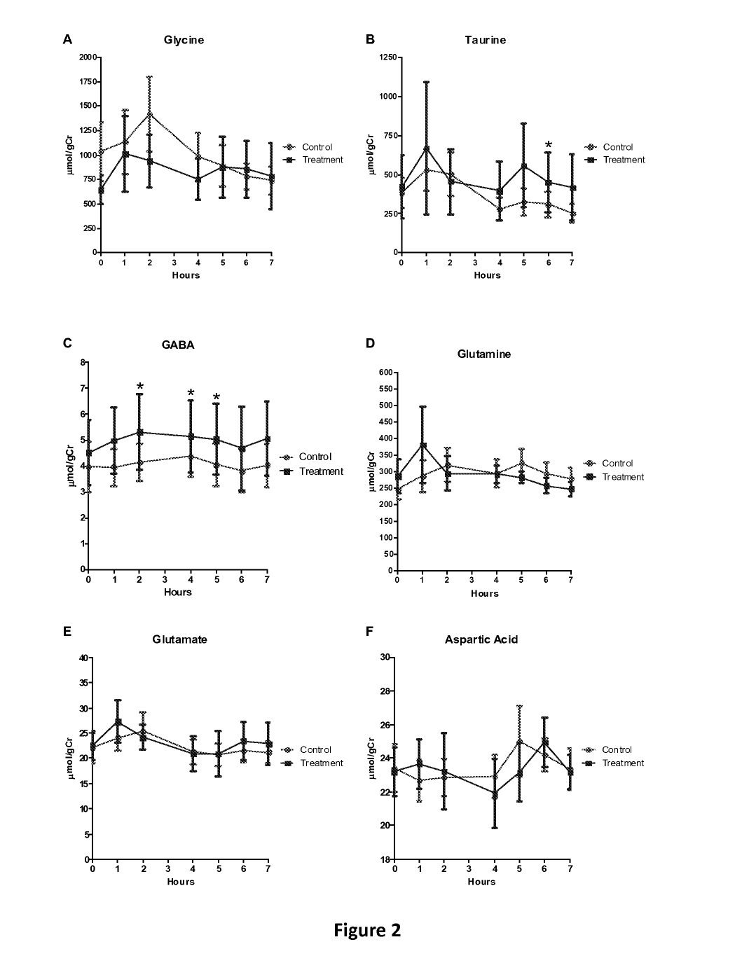

Levels of amino acids such as glycine, taurine, GABA, glutamine, glutamate or aspartic acid were also assessed. Glycine levels decreased at 1, 2, 4, and 5 hours, but increased slightly at 6 and 7 hours (FIG. 2, A). No statistical significance was observed at any time point (Table 1). Taurine levels increased at 1, 4, 5, 6, and 7 hours, but decreased at 2 hours (FIG. 2, B). Statistical significance was observed at the 6 hour time point (p<0.05; Table 1). Increased GABA levels were observed at all time points and significance was observed at 2, 4 and 5 hours (FIG. 2, C; p<0.05, Table 1). Glutamine levels after treatment were very similar to control levels with a slight increase at 1 hour, decreases at 2, 5, 6, and 7 hours, and no significance observed (FIG. 2, D; Table 1). Changes in glutamate, after treatment, were also very slight with increases observed at 1, 6, and 7 hours, decreases at 2, and 4 hours, and no significance at any time point (FIG. 2, E; Table 1). Aspartic acid levels increased slightly at 1, 2, and 6 hours and decreased at 4, 5, and 7 hours, but no statistical significance was observed (FIG. 2, F; Table 1).

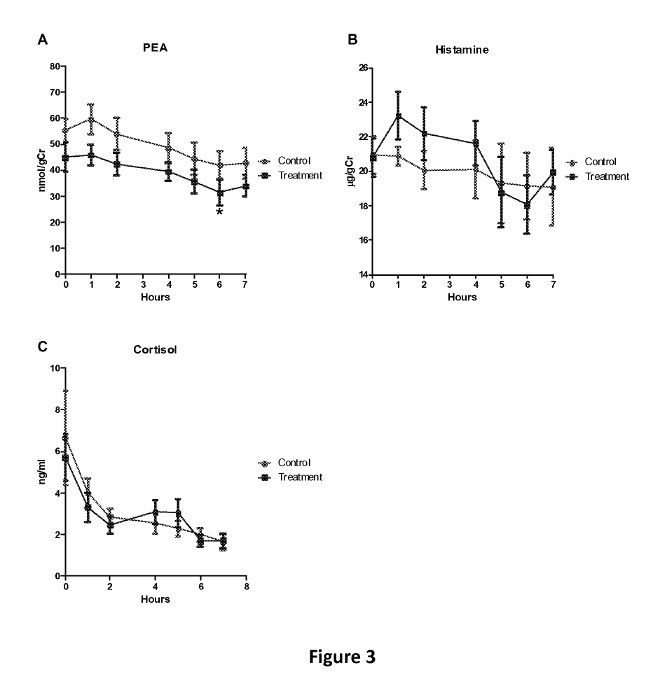

The effects the test composition on PEA and histamine were also determined. Decreased PEA levels were observed at all time points, but only the decrease at 6 hours was statistically significant (FIG. 3, A; Table 1). Histamine values increased at the 1, 2, 4 and 7 hour time points, but decreased slightly at 5 and 6 hours with no significance observed (FIG. 3, B; Table 1).

Evidence suggests that CNS acetylcholine neurons can modulate adrenal sympathetic activity by stimulating cortisol release from the adrenal glands. Pavlov et al., Mol. Med., 9: 125-134 (2003). Cortisol levels from both the control day and treatment day showed circadian rhythm; however, cortisol levels after treatment were not significantly different from control levels (Table 1). Slightly decreased levels were observed relative to control samples at 1, 2, and 6 hours and slightly increased levels were seen at 4 and 5 hours (FIG. 3, C).

TABLE 1
The p values for each neurotransmitter and cortisol from
a one-tailed t-test on the means of paired samples.

| | 0 hr | 1 hr | 2 hr | 4 hr | 5 hr | 6 hr | 7 hr |
|---|---|---|---|---|---|---|---|
| Epinephrine | 0.0411 | 0.4349 | 0.1236 | 0.2378 | 0.1426 | 0.1032 | 0.2369 |
| Norepinephrine | 0.3760 | 0.1130 | 0.1395 | 0.0657 | 0.0315 | 0.0267 | 0.4644 |
| Dopamine | 0.0364 | 0.3013 | 0.3609 | 0.1815 | 0.3724 | 0.4264 | 0.4886 |
| DOPAC | 0.0079* | 0.0078* | 0.0017* | 0.0034* | 0.0510 | 0.0223 | 0.0056* |
| Serotonin | 0.2455 | 0.2226 | 0.3983 | 0.1057 | 0.0002* | 0.2917 | 0.0028* |
| 5-HIAA | 0.2049 | 0.2498 | 0.3495 | 0.2334 | 0.1520 | 0.2592 | 0.4280 |
| Glycine | 0.2209 | 0.1079 | 0.3270 | 0.4514 | 0.1664 | 0.1482 | 0.1280 |
| Taurine | 0.4961 | 0.3818 | 0.3809 | 0.2850 | 0.0686 | 0.0434 | 0.0725 |
| GABA | 0.1684 | 0.0862 | 0.0155 | 0.0142 | 0.0137 | 0.0895 | 0.0512 |
| Glutamine | 0.1128 | 0.1747 | 0.3531 | 0.0519 | 0.3856 | 0.2417 | 0.4827 |
| Glutamate | 0.4589 | 0.1556 | 0.3410 | 0.3254 | 0.3192 | 0.1438 | 0.0905 |
| Aspartic Acid | 0.4545 | 0.2486 | 0.4705 | 0.1485 | 0.2495 | 0.3972 | 0.4153 |
| PEA | 0.1175 | 0.0621 | 0.1142 | 0.0604 | 0.1540 | 0.0175 | 0.0934 |
| Histamine | 0.4244 | 0.1023 | 0.0663 | 0.0674 | 0.0886 | 0.3647 | 0.2621 |
| Cortisol | 0.2726 | 0.1367 | 0.1079 | 0.1966 | 0.1438 | 0.0938 | 0.3453 |

The p values <0.05 are in bold type. The p values <0.01 are marked by an asterisk. The p values approaching significance are in italics type.

### Cytokines

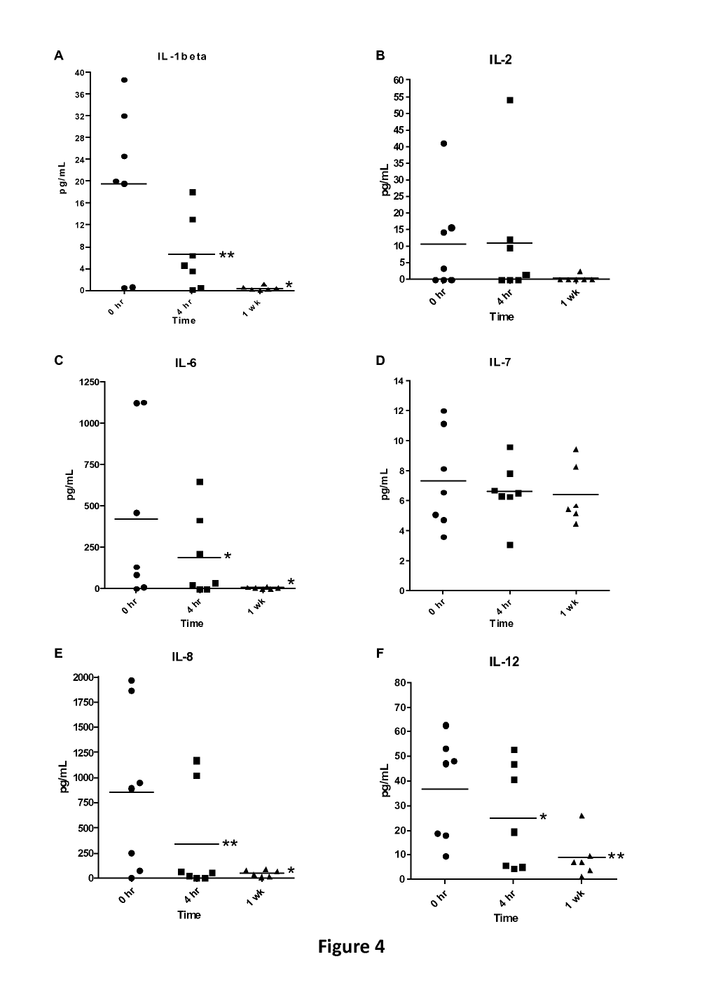

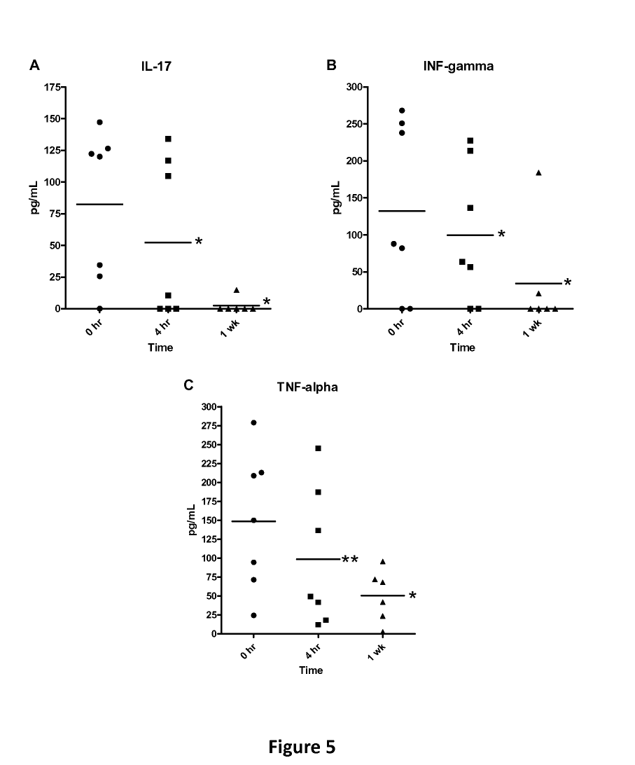

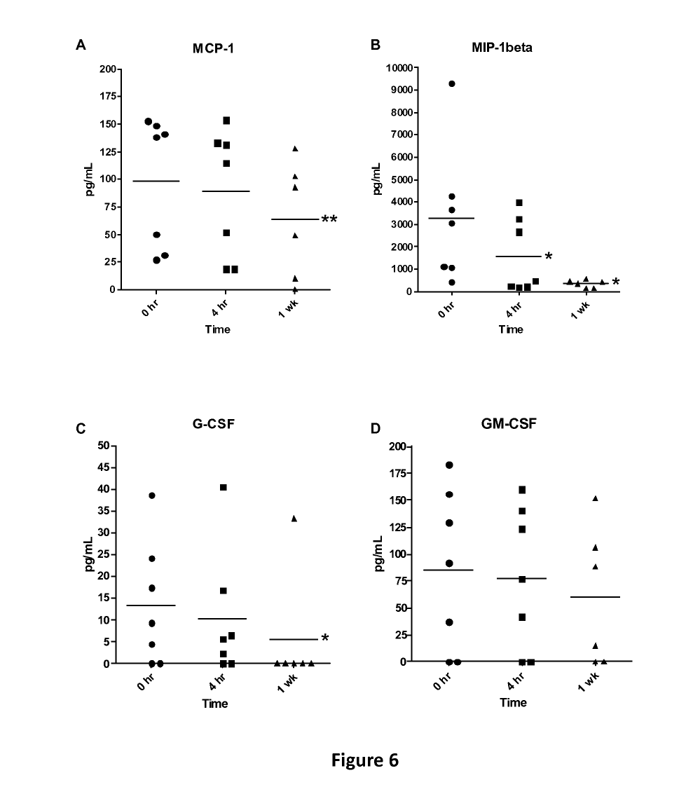

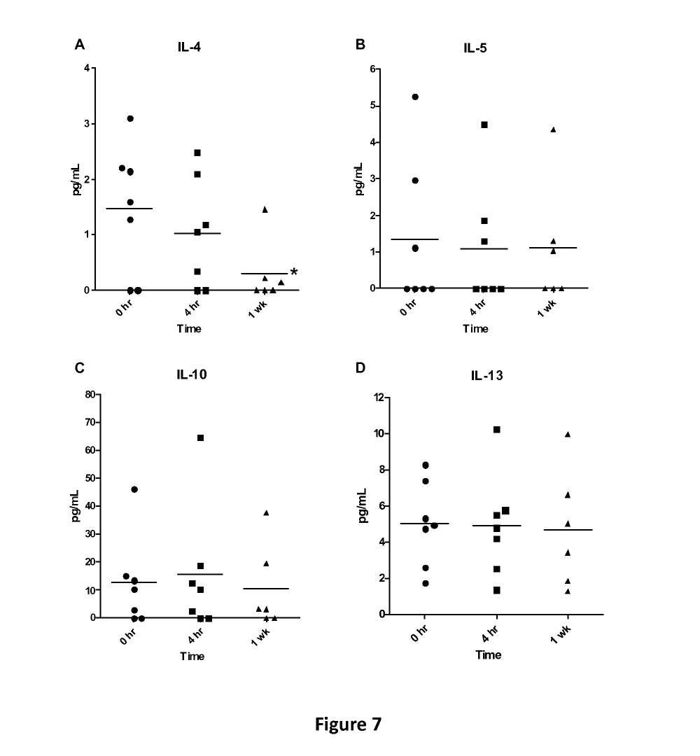

Acetylcholine appears to play a negative regulatory effect on inflammation by reducing the production of cytokines from macrophages. Wang et al., Nature, 421: 384-388 (2003). The levels of several pro-inflammatory and anti-inflammatory cytokines, chemokines, and growth factors in serum were measured from seven subjects, collected before (0 hour) and 4 hours after oral ingestion of the test composition. Significant decreases in several pro-inflammatory cytokines, such as IL-1β (p<0.01), IL-6 (p<0.05), IL-8 (p<0.01), IL-12 (p<0.05), IL-17 (p<0.05), IFN-γ (p<0.05) and TNF-α (p<0.01), were observed at 4 hours (Table 2; FIG. 4, A, C, E, F and FIG. 5, A, B, C, respectively). The pro-inflammatory cytokines IL-2 and IL-7 did not show significant changes after 4 hours (Table 2; FIG. 4, B, D). Changes in the pro-inflammatory chemokines and growth factors were also observed. MCP-1 showed a slight decrease at 4 hours and MIP-1β levels decreased significantly at 4 hours (p<0.05) (Table 2; FIG. 6, A, B). Both G-CSF and GM-CSF levels decreased after 4 hours of treatment (Table 2; FIG. 6, C, D), but neither decreased significantly. The anti-inflammatory cytokines IL-5, IL-10 and IL-13 were unaffected by the test composition treatment (Table 2; FIG. 7, B, C, D); only IL-4 showed a non-significant decrease at 4 hours (Table 2; FIG. 7, A).

TABLE 2
p values for each immune marker from a one-tailed
t-test on the means of paired samples.

| | |
|---|---|
| 4 hr | 1 week |

Pro-inflammatory

| | | |
|---|---|---|
| IL-1b | 0.0044* | 0.0173 |
| IL-2 | 0.4283 | 0.1028 |
| IL-6 | 0.0324 | 0.0435 |
| IL-7 | 0.1805 | 0.4361 |
| IL-8 | 0.0070* | 0.0373 |
| IL-12 | 0.0418 | 0.0073* |
| IL-17 | 0.0381 | 0.0148 |
| TNFa | 0.0086* | 0.0123 |
| INF-g | 0.0339 | 0.0443 |

Chemokines

| | | |
|---|---|---|
| MCP-1 | 0.0862 | 0.0074* |
| MIP-1b | 0.0364 | 0.0374 |

Growth Factors

| | | |
|---|---|---|
| G-CSF | 0.0571 | 0.0366 |
| GM-CSF | 0.1201 | 0.1119 |

Anti-inflammatory

| | | |
|---|---|---|
| IL-4 | 0.0509 | 0.0211 |
| IL-5 | 0.1196 | 0.0899 |
| IL-10 | 0.1575 | 0.2622 |
| IL-13 | 0.4385 | 0.4424 |

The p values < 0.05 are in bold type. The p values < 0.01 are marked by an asterisk. The p values approaching significance are in italics type.

### Heart Rate Variability

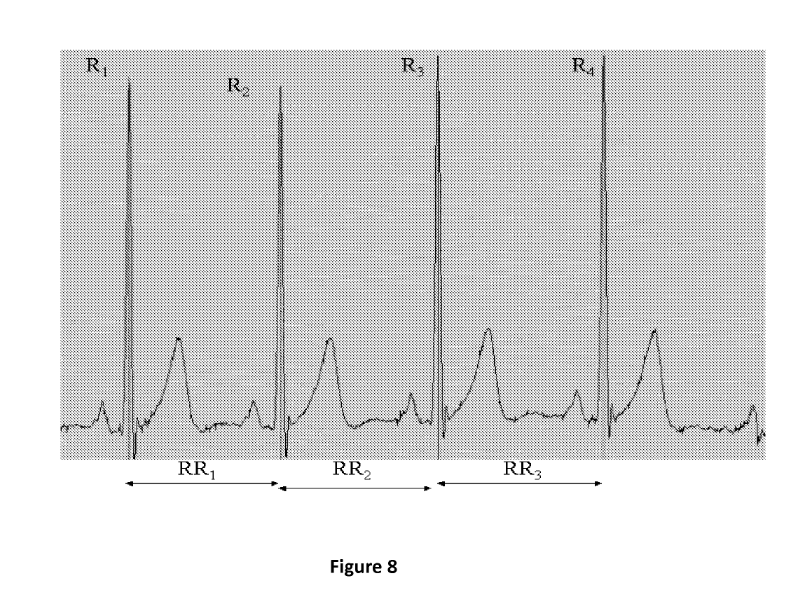

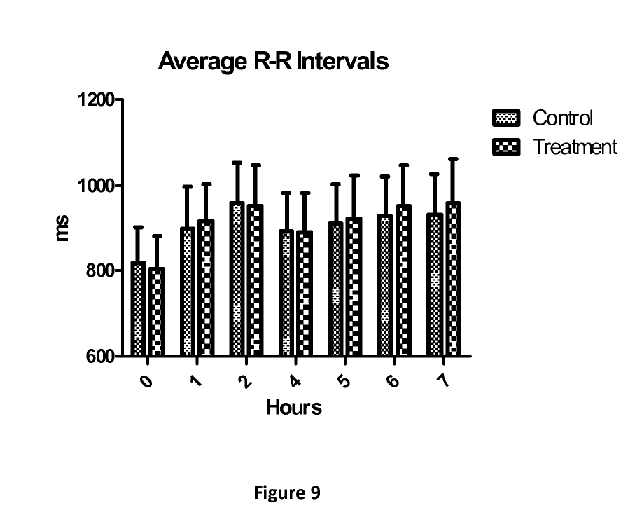

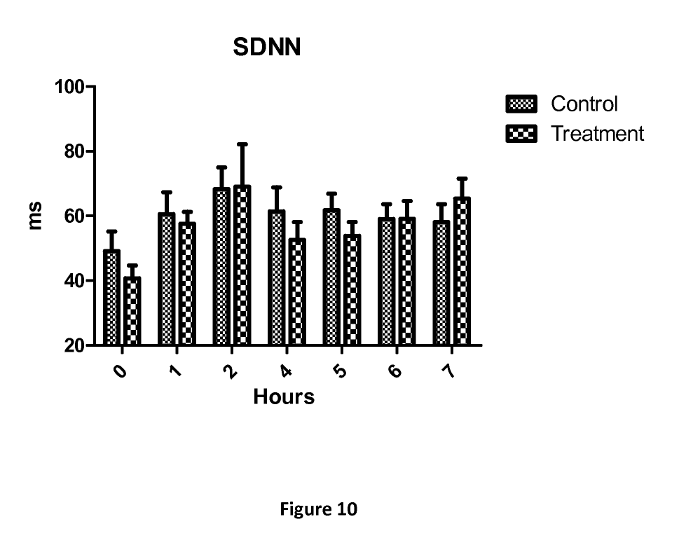

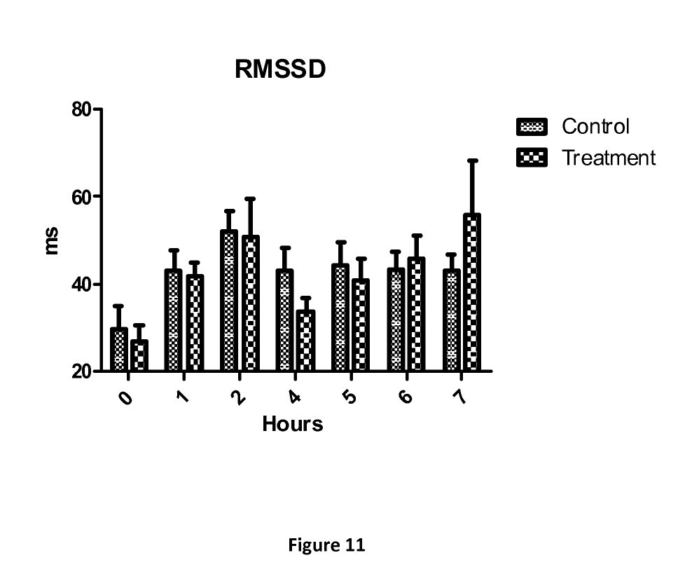

In addition to acetylcholine vagal activity in the regulation of immunological pathways, regulation of heart rate is another primary function. The sympathetic nervous system increases heart rate through noradrenergic postganglionic neurons to stimulate the sinoatrial node (SA node) which leads to atrial contraction, whereas the parasympathetic nervous system can utilize acetylcholine from the vagal nerve to decrease heart rate through the inhibition of the SA node. Pieper & Hammill, Mayo Clin. Proc., 70:955-964 (1995). In order to investigate whether treatment with the test composition affected heart rate variability (HRV), each subject's heart rate was measured on a control day and treatment day at baseline, 1 hour, 2 hours, 4 hours, 5 hours, 6 hours, and 7 hours. The time domain measures of HRV are calculated by statistical analysis (means and variance) from the lengths of successive R-R intervals. Kleiger et al., Ann. Noninvasive. Electroardiol., 10: 88-101 (2005). R-R intervals are a measure of the time duration between two consecutive R waves of the ECG (FIG. 8). Studies have shown that a decrease in parasympathetic activity results in a decrease in time domain measures of HRV. Hayano et al., Am J. Cardiol., 67:199-204 (1991). The average R-R interval, SDNN, and RMSSD for each time point were calculated and analyzed. Statistically significant changes were found for the time measure of the average R-R intervals after treatment (p<0.01), however, the control and treatment days were not significantly different (FIG. 9). Statistically significant changes were also found for the time measure of SDNN after treatment (p<0.01), however, the control and treatment days were not significantly different (FIG. 10). Finally, statistically significant changes were found for the time measure of RMSSD after treatment (p<0.01), however, the control and treatment days were not significantly different (FIG. 11).

### Extended Use of Test Composition

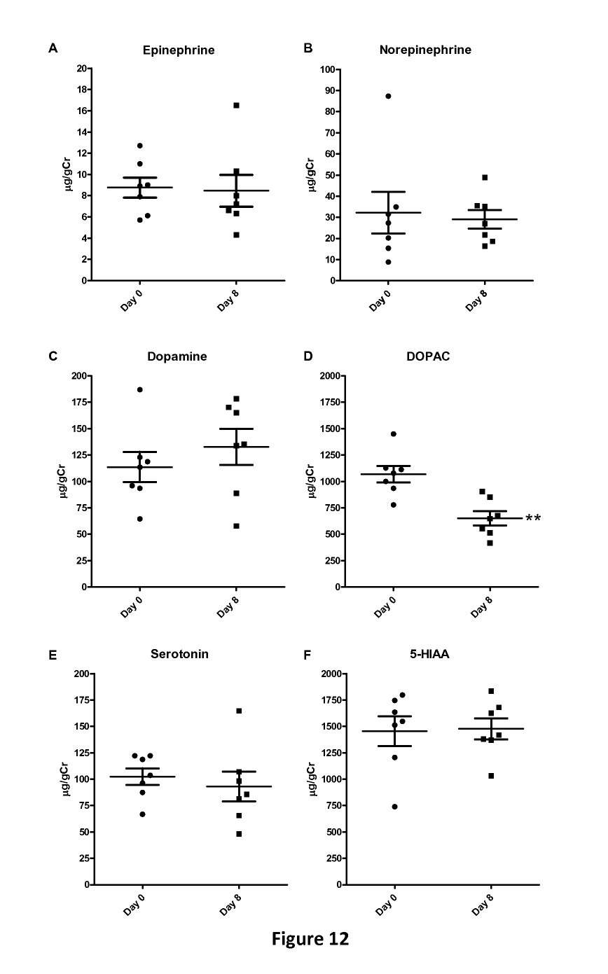

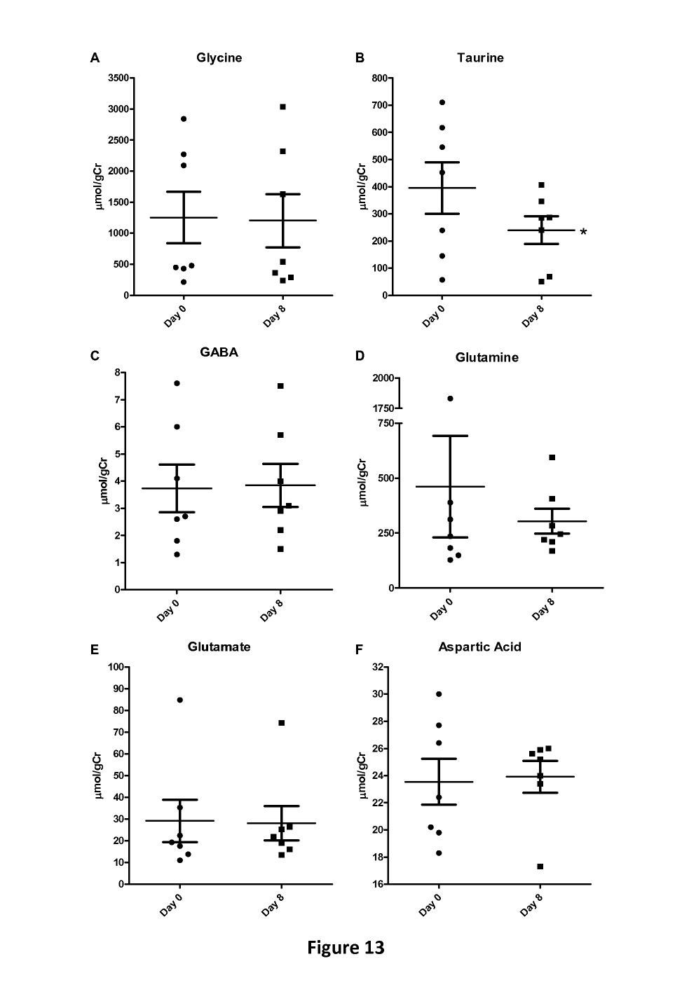

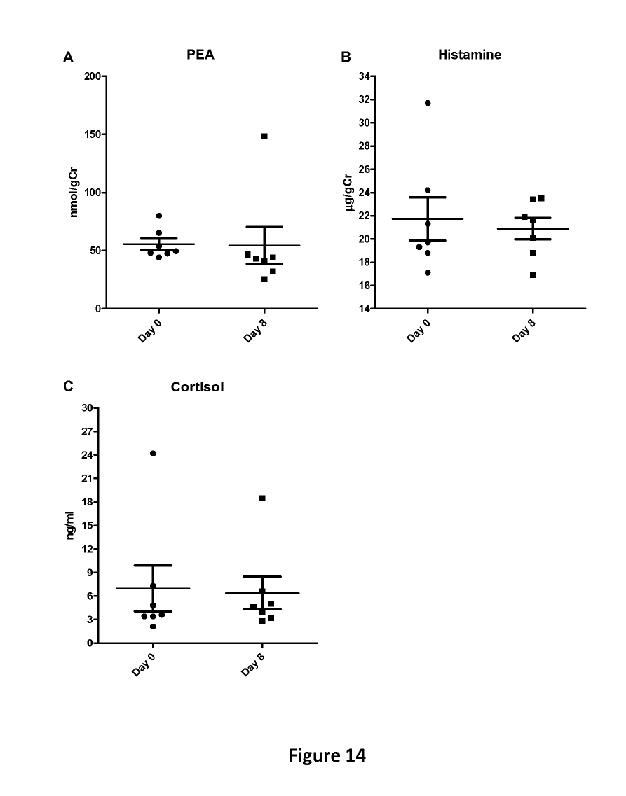

To determine the effects of the test composition on neurotransmitters, cortisol, and cytokines over a longer time period, the subjects orally ingested the test composition twice daily for six additional days (after Day 1) and then collected specimens on Day 8. As explained above, the sample size was n=7 for the data from Day 8. The values for each subject and the means with the standard error of the mean (SEM) for each neurotransmitter and cortisol were graphed (FIGS. 12, 13, 14). The data from urine and saliva specimens collected at 0 hour on Day 0 were compared to specimens collected on Day 8. Statistical analysis was performed using the one-tailed t-test for the means of paired samples (Table 3). Significant decreases were observed for DOPAC (p<0.01) and taurine (p<0.05) (Table 3; FIG. 12, D and FIG. 13, B, respectively). No other significant changes were observed for other neurotransmitters or cortisol (FIGS. 12, 13, 14). Significant decreases were also seen after 1 week for IL-1β (p<0.05), IL-6 (p<0.05), IL-8 (p<0.05), IL-12 (p<0.01), IL-17 (p<0.05), IFN-γ (p<0.05) and TNF-α (p<0.05) when compared to 0 hour serum specimens collected on Day 1 (Table 2; FIG. 4, A, C, E, F and FIG. 5, A, B, C, respectively). IL-2 levels did decrease after 1 week, but did not show statistical significance (FIG. 14 B). IL-7 levels did not change after 1 week of treatment (FIG. 4, D). MCP-1 and MIP-1β showed significant decreases after 1 week (p<0.01 and p<0.05, respectively) (Table 2; FIG. 6, A, B). In addition, G-CSF and GM-CSF levels decreased after 1 week of treatment (FIG. 6, C, D), but only GM-CSF levels decreased significantly (p<0.05) (Table 2). Only the anti-inflammatory cytokine IL-4 showed a statistical significant decrease at 1 week (FIG. 7, A, Table 2). IL-5, IL-10 and IL-13 were unaffected by the test composition treatment (FIG. 7, B, C, D).

TABLE 3
The p values for each neurotransmitter and cortisol from
a one-tailed t-test of the means of paired samples.

| | Day 8 |
|---|---|
| Epinephrine | 0.3922 |
| Norepinephrine | 0.3777 |
| Dopamine | 0.1035 |
| DOPAC | 0.0011* |
| Serotonin | 0.2046 |
| 5-HIAA | 0.3798 |
| Glycine | 0.3337 |
| Taurine | 0.0207 |
| GABA | 0.4113 |
| Glutamine | 0.2100 |
| Glutamate | 0.3401 |
| Aspartic Acid | 0.4122 |
| PEA | 0.4679 |
| Histamine | 0.2785 |
| Cortisol | 0.4357 |

The p values < 0.05 are in bold type. The p values < 0.01 are marked with an asterisk.

### Positive Outcomes And Side Effects

Subjects reported the following observations regarding positive changes in physical or psychological symptoms: feeling fine/normal, feeling more clear-headed, feeling awake and more alert; having good energy, having less frequent headaches, having energy and focus without caffeine, and having more mental energy; drinking less caffeine; and waking refreshed. Reports of negative changes included the following observations: headache, light-headedness, trouble concentrating, brain fog, stomachache, fatigue, and lack of focus.

As a whole, these data suggest that oral administration of a composition as described herein may decrease urinary norepinephrine, DOPAC, and PEA levels, decrease serum pro-inflammatory cytokines, and increase urinary serotonin, taurine, and GABA. These findings support a conclusion that the test composition enhances acetylcholine function as shown by decreased norepinephrine and pro-inflammatory cytokines and increased serotonin. The test composition may support acetylcholine function and provide relief to individuals with an excessive inflammatory response.

### Other Embodimants

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A composition comprising a therapeutically effective amount of two or more of an anticholinesterase compound, a choline compound, and a carnitine compound.

2. The composition of claim 1, wherein the composition comprises an anticholinesterase compound and a choline compound.

3. The composition of claim 1, wherein the composition comprises an anticholinesterase compound a carnitine compound.

4. The composition of claim 1, wherein the composition comprises a choline compound, and a carnitine compound.

5. The composition of claim 1, wherein the composition consists of an anticholinesterase compound, a choline compound, and a carnitine compound.

6. The composition of claim 1, wherein the anticholinesterase compound comprises huperzine A, or a pharmaceutically acceptable salt thereof.

7. The composition of claim 6, wherein the therapeutically effective amount of huperzine A is 0.003 mg to 3.0 mg.

8. The composition of claim 1, wherein the choline compound comprises alpha-GPC, or a pharmaceutically acceptable salt thereof.

9. The composition of claim 8, wherein the therapeutically effective amount of alpha-GPC is 10 mg to 10,000 mg.

10. The composition of claim 1, wherein the carnitine compound comprises acetyl-L-carnitine, or a pharmaceutically acceptable salt thereof.

11. The composition of claim 10, wherein the therapeutically effective amount of acetyl-L-carnitine is 20.0 mg to 20,000 mg.

12. A kit comprising a therapeutically effective amount of two or more of an anticholinesterase compound, a choline compound, and a carnitine compound in separate dosage forms.

13. A method of regulating one of more inflammatory pathways in a human, wherein the method comprises administering to the human an effective amount of the composition of claim 1.

14. The method of claim 13, wherein regulating one or more inflammatory pathways in the human comprises altering a level of one or more inflammatory mediators.

15. The method of claim 14, wherein one or more inflammatory mediators are selected from the group consisting of cytokines, chemokines, growth factors, neurotransmitters, and amino acids.

16. The method of claim 15, wherein one or more inflammatory mediators are selected from the group consisting of IL-1α, IL-1β, IL-6, TNF-α, leukocyte inhibitory factor (LIF), INF-γ, ciliary neuronotrophic factor (CNTF), GM-CSF, IL-11, IL-12, IL-17, IL-18, IL-8, IL-4, IL-10, IL-13, IL-16, IFN-α, G-CSF, TGF-β, soluble receptors for TNF and IL-6, norepinephrine, epinephrine, serotonin, and dopamine.

17. The method of claim 15, wherein the level of one or more pro-inflammatory mediators is decreased relative to the level of anti-inflammatory mediators.

18. The method of claim 13, wherein the human is identified as being obese.

19. The method of claim 13, wherein the human is identified as having one or more of the group selected from: autism, Lyme disease, irritable bowel syndrome, a food allergy, coronary artery disease, a mood disorder and hyperthyroidism.

20. A method of treating obesity in a human, wherein the method comprises administering to the human an effective amount of the composition of claim 1.

21. A method of supporting a healthy body weight in a human, wherein the method comprises administering to the human an effective amount of the composition of claim 1.

22. A method of treating autism in a human, wherein the method comprises administering to the human an effective amount of the composition of claim 1.

23. A method of treating panic attacks in a human, wherein the method comprises administering to the human an effective amount of the composition of claim 1.

24. A method of treating sleep apnea in a human, wherein the method comprises administering to the human an effective amount of the composition of claim 1.

25. A method of supporting healthy sleep patterns in a human, wherein the method comprises administering to the human an effective amount of the composition of claim 1.

26. A method of treating coronary artery disease in a human, wherein the method comprises administering to the human an effective amount of the composition of claim 1.

27. A method of supporting a healthy cardiovascular system in a human, wherein the method comprises administering to the human an effective amount of the composition of claim 1.

28. A method of treating Lyme disease in a human, wherein the method comprises administering to the human an effective amount of the composition of claim 1.

29. A method of treating irritable bowel syndrome in a human, wherein the method comprises administering to the human an effective amount of the composition of claim 1.

30. A method of treating gluten sensitivity in a human, wherein the method comprises administering to the human an effective amount of the composition of claim 1.

31. A method of promoting healthy digestive functioning in a human, where the method comprises administering to the human an effective amount of the composition of claim 1.

32. A method of treating hyperthyroidism in a human, wherein the method comprises administering to the human an effective amount of the composition of claim 1.

33. A method of restoring balanced activity of the thyroid gland in a human, wherein the method comprises administering to the human an effective amount of the composition of claim 1.

34. A method of altering the level of a neurotransmitter in a human, comprising administering to the human the composition of claim 1.

35. A method of altering the level of a cytokine in a human, comprising administering to the human the composition of claim 1.

36. A method of altering the level of a growth factor in a human comprising, administering to the human comprising administering to the human the composition of claim 1.

37. A method of treating excess inflammation in a human, wherein the method comprises administering to the human an effective amount of the composition of claim 1.

38. A method of treating one or more of ADHD, ADD, long-term depression (‘dysthymic’ depression) and PTSD, comprising administering to the human the composition of claim 1.

39. The method of claim 38, wherein the composition supports central nervous system cholinergic activities that target the locus ceruleus and the rostral ventral lateral medulla of the brain stem and/or increases the activity of the HPA axis via stimulation of the LC/brainstem nuclei.
